(19) United States
(12) Patent Application Publication
(10) Pub. No.: US 2008/0113025 A1
(43) Pub. Date: May 15, 2008

(54) COMPOSITIONS COMPRISING NANOPARTICULATE NAPROXEN AND CONTROLLED RELEASE HYDROCODONE

(75) Inventors: John G. Devane, Athlone (IE); Paul Stark, Glasson (IE); Niall Fanning, Dublin (IE); Gurvinder Singh Rekhi, Suwanee, GA (US); Scott A. Jenkins, Downington, PA (US); Gary Liversidge, Westchester, PA (US)

Correspondence Address: Fox Rothschild, LLP, P.O. Box 431, Lansdale, PA, 19446-0431, US

(73) Assignee: Elan Pharma International Limited, Athlone (IE)

(21) Appl. No.: 11/768,154

(22) Filed: Jun. 25, 2007

Related U.S. Application Data
(63) Continuation-in-part of application No. 11/372,857, filed on Mar. 10, 2006.
Application No. 11/372,857 is a continuation-in-part of application No. 10/827,689, filed on Apr. 19, 2004.
Application No. 10/827,689 is a continuation of application No. 10/354,483, filed on Jan. 30, 2003, now Pat. No. 6,793,936.
Application No. 10/354,483 is a continuation of application No. 10/331,754, filed on Dec. 30, 2002, now Pat. No. 6,902,742.
Application No. 10/331,754 is a continuation of application No. 09/850,425, filed on May 7, 2001, now Pat. No. 6,730,325.
Application No. 09/850,425 is a continuation of application No. 09/566,636, filed on May 8, 2000, now Pat. No. 6,228,398.
Application No. 09/566,636 is a continuation of application No. PCT/US99/25632, filed on Nov. 1, 1999.
(60) Provisional application No. 60/815,885, filed on Jun. 23, 2006.
(60) Provisional application No. 60/106,726, filed on Nov. 2, 1998.

(51) Int. Cl. A61K 31/485; A61K 9/26

(52) U.S. Cl. 424/470; 514/282

(57) ABSTRACT

The invention relates to a compositions comprising a nanoparticulate naproxen composition in combination with a multiparticulate modified release hydrocodone composition that, upon administration to a patient, delivers a hydrocodone in a bimodal or multimodal manner. The multiparticulate modified release composition comprises a first component and at least one subsequent component; the first component comprising a first population of hydrocodone-comprising particles and the at least one subsequent component comprising a second population of hydrocodone-comprising particles, wherein the combination of the components exhibit a bimodal or multimodal release profile. The invention also relates to a solid oral dosage form comprising such a combination composition.

### CROSS-REFERENCE TO RELATED APPLICATIONS

This application claims the benefit of provisional Application No. 60/815,885, filed Jun. 23, 2006, and this application is a continuation-in-part of application Ser. No. 11/372,857, filed Mar. 10, 2006, is a continuation-in-part of application Ser. No. 10/827,689, filed Apr. 19, 2004, which is a continuation of application Ser. No. 10/354,483, filed Jan. 30, 2003, now U.S. Pat. No. 6,793,936, which is a continuation of application Ser. No. 10/331,754, filed Dec. 30, 2002, now U.S. Pat. No. 6,902,742, which is a continuation of application Ser. No. 09/850,425, filed May 7, 2001, now U.S. Pat. No. 6,730,325, which is a continuation of application Ser. No. 09/566,636, filed May 8, 2000, now U.S. Pat. No. 6,228,398, which is a continuation of Application No. PCT/US99/25632, filed Nov. 1, 1999, which claims the benefit of provisional Application No. 60/106,726, filed Nov. 2, 1998. All of the above-identified applications are hereby incorporated by reference.

### FIELD OF INVENTION

The present invention relates to compositions comprising nanoparticulate naproxen, or a salt or derivative thereof, in combination with a multiparticulate modified release composition comprising hydrocodone, or a salt or derivative thereof. In particular the present invention relates to compositions comprising nanoparticulate naproxen, or a salt or derivative thereof, in combination with a multiparticulate modified release composition that in operation delivers hydrocodone, or a salt or derivative thereof, in a bimodal or multimodal manner. The present invention further relates to solid oral dosage forms comprising such multiparticulate controlled release compositions as well as methods for delivering such compositions to a patient in need thereof.

### BACKGROUND OF INVENTION

### A. Background Regarding Oral Controlled Release Compositions

The effectiveness of pharmaceutical compounds in the prevention and treatment of disease states depends on a variety of factors including the rate and duration of delivery of the compound from the dosage form to the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective drug therapies.

The release profiles of dosage forms may exhibit different rates and durations of release and may be continuous or pulsatile. Continuous release profiles include release profiles in which one or more pharmaceutical compounds are released continuously, either at a constant or variable rate, and pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. Similar to the variables applicable to the release profile, the associated plasma profile in a patient may exhibit constant or variable blood plasma concentration levels of the pharmaceutical compounds in the dosage form over the duration of action and may be continuous or pulsatile. Continuous plasma profiles include plasma profiles of all rates and duration which exhibit a single plasma concentration maximum. Pulsatile plasma profiles include plasma profiles in which at least two higher blood plasma concentration levels of pharmaceutical compound are separated by a lower blood plasma concentration level. Pulsatile plasma profiles exhibiting two peaks may be described as “bimodal.”

When two or more components of a dosage form have different release profiles, the release profile of the dosage form as a whole is a combination of the individual release profiles. The release profile of a two-component dosage form in which each component has a different release profile may described as “bimodal.” For dosage forms of more than two components in which each component has a different release profile, the resultant release profile of the dosage form may be described as “multimodal.” Depending on, at least in part, the pharmacokinetics of the pharmaceutical compounds that are used as well as the specific release profiles of the components of the dosage form, a bimodal or multimodal release profile may result in either a continuous or a pulsatile plasma profile in a patient. Conventional frequent dosage regimes in which an immediate release (IR) dosage form is administered at periodic intervals typically gives rise to a pulsatile plasma profile. In such cases, a peak in the plasma drug concentration is observed after administration of each IR dose with troughs (regions of low drug concentration) developing between consecutive administration time points. Such dosage regimes (and their resultant pulsatile plasma profiles) can have particular pharmacological and therapeutic effects associated with them that are beneficial for certain drug therapies. For example, the wash out period provided by the fall off of the plasma concentration of the active ingredient between peaks has been thought to be a contributing factor in reducing or preventing patient tolerance to various types of drugs.

Many controlled release drug formulations are aimed at producing a zero-order release of the drug compound. Indeed, it is often a specific object of these formulations to minimize the peak-to-trough variation in plasma concentration levels associated with conventional frequent dosage regimes. For certain drugs, however, some of the therapeutic and pharmacological effects intrinsic in a pulsatile system may be lost or diminished as a result of the constant or nearly constant plasma concentration levels achieved by zero-order release drug delivery systems. Thus, modified release compositions or formulations which substantially mimic the release of frequent IR dosage regimes, while reducing the need for frequent dosing, is desirable. Similarly, modified release compositions or formulations which combine the benefits of at least two different release profiles to achieve a resultant plasma profile exhibiting phatinacokinetic values within therapeutically effective parameters is also desirable.

Shah et al., J Cont. Rel. (1989) 9:169-175 purports to disclose that certain types of hydroxypropyl methylcellulose ethers compressed into a solid dosage form with a therapeutic agent may produce a bimodal release profile. However, it is noted that while polymers from one supplier yielded a bimodal profile, the same polymers with almost identical product specifications obtained from a different source gave non-bimodal release profiles.

Giunchedi et al., Int. J. Pharm (1991) 77:177-181 discloses the use of a hydrophilic matrix multiple-unit formulation for the pulsed release of ketoprofen. Giunchedi et al. teach that ketoprofen is rapidly eliminated from the blood after dosing (plasma half-life 1-3 hours) and consecutive pulses of drug may be more beneficial than constant release for some treatments. The multiple-unit formulation disclosed comprises four identical hydrophilic matrix tablets placed in a gelatin capsule. Although the in vivo studies show two peaks in the plasma profile there is no well defined wash out period and the variation between the peak and trough plasma levels is small.

Conte et al., Drug Dev. Ind. Pharm, (1989) 15:2583-2596 and EP 0 274 734 (Phamidea Sri) teach the use of a three layer tablet for delivery of ibuprofen in consecutive pulses. The three layer tablet is made up of a first layer containing the active ingredient, a barrier layer (the second layer) of semi-permeable material which is interposed between the first layer and a third layer containing an additional amount of active ingredient. The barrier layer and the third layer are housed in an impermeable casing. The first layer dissolves upon contact with a dissolving fluid while the third layer is only available after dissolution or rupture of the barrier layer. In such a tablet the first portion of active ingredient must be released instantly. This approach also requires the provision of a semi-permeable layer between the first and third layers in order to control the relative rates of delivery of the two portions of active ingredient. Additionally, rupture of the semi-permeable layer leads to uncontrolled dumping of the second portion of the active ingredient which may not be desirable.

U.S. Pat. No. 5,158,777 (E. R. Squibb & Sons Inc.) discloses a formulation comprising captopril within an enteric or delayed release coated pH stable core combined with additional captopril which is available for immediate release following administration. In order to form the pH stable core, chelating agents such as disodium edetate or surfactants such as polysorbate 80 are used either alone or in combination with a buffering agent. The compositions have an amount of captopril available for immediate release following oral administration and an additional amount of pH stabilized captopril available for release in the colon.

U.S. Pat. Nos. 4,728,512, 4,794,001 and 4,904,476 (American Home Products Corp.) relate to preparations providing three distinct releases. The preparation contains three groups of spheroids containing an active medicinal substance: the first group of spheroids is uncoated and rapidly disintegrates upon ingestion to release an initial dose of medicinal substance; the second group of spheroids is coated with a pH sensitive coat to provide a second dose; and the third group of spheroids is coated with a pH independent coat to provide to third dose. The preparation is designed to provide repeated release of medicinal substances which are extensively metabolized presystemically or have relatively short elimination half-lives.

U.S. Pat. No. 5,837,284 (Mehta et al) discloses a methylphenidate dosage form having immediate release and delayed release particles. The delayed release is provided by the use of ammonio methacrylate pH independent polymers combined with certain fillers.

### B. Background Regarding Hydrocodone

A typical example of a drug which may produce tolerance in patients is hydrocodone. Hydrocodone or dihydrocodeinone (marketed as Vicodin®, Anexsia®, Dicodid®, Hycodan®, Hycomine®, Lorcet®, Lortab®, Norco®, Hydroco®, Tussionex®, and Vicoprofen®), also known as 4,5a-Epoxy-3-methoxy-17-methylmorphinan-6-one tartrate (1:1) hydrate (2:5), is an opiod derived from either of the naturally occurring opiates codeine or thebaine. The compound has the following structure:

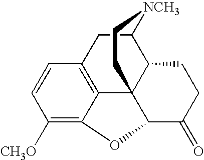

Hydrocodone has the chemical formula C₁₈H₂₁N_{O3}, a molecular weight of 299.368, and a half life of 4-8 hours. Hydrocodone is an orally active narcotic analgesic and antitussive. Sales and production of this drug have increased significantly in recent years, as have diversion and illicit use. Hydrocodone is commonly available in tablet, capsule and syrup form.

As a narcotic, hydrocodone relieves pain by binding to opioid receptors in the brain and spinal cord. It may be taken with or without food. When taken with alcohol, it can intensify drowsiness. It may interact with monoamine oxidase inhibitors, as well as other drugs that cause drowsiness. Common side effects include dizziness, lightheadedness, nausea, drowsiness, euphoria, vomiting, and constipation. Some less common side effects are allergic reaction, blood disorders, changes in mood, mental fogginess, anxiety, lethargy, difficulty urinating, spasm of the ureter, irregular or depressed respiration and rash.

Hydrocodone can be habit-forming, and can lead to physical and psychological addiction. In the U.S., pure hydrocodone and forms containing more than 15 mg per dosage unit are considered Schedule II drugs. Those containing less than or equal to 15 mg per dosage unit in combination with acetaminophen or another non-controlled drug are called Hydrocodone Compounds and are considered Schedule III drugs. Hydrocodone can be found in combination with other drugs such as paracetamol (acetaminophen), aspirin, ibuprofen and homatropine methylbromide.

The presence of acetaminophen in hydrocodone-containing products deters many drug users from taking excessive amounts. However, some users will get around this by extracting a portion of the acetaminophen using hot/cold water, taking advantage of the water-soluble element of the drug. It is not uncommon for addicts to have liver problems from consuming excessive amounts of acetaminophen over a long period of time; taking 10,000 to 15,000 miligrams of acetaminophen in a period of 24 hours typically results in severe hepatoxicity, and doses in the range of 15,000-20,0000 miligrams a day have been reported as fatal. It is this factor that leads many addicts to use only single entity opiates such as Oxycontin.

Daily consumption of hydrocodone should not exceed 40 milligrams in patients not tolerant to opiates. However, it clearly states in the 2006 PDR (Physicians Desk Reference) that Norco 10, containing 10 miligrams of hydrocodone and 325 miligrams of Apap, can be taken at a dosage of up to twelve tablets per day (120 miligrams of hydrocodone). Such high amounts of hydrocodone are only intended for opiate tolerant patients, and titration to such levels must be monitored very carefully. This restriction is only limited by the fact that twelve tablets, each containing 325 miligrams of Apap, puts the patient right below the 24 hour FDA maximum of 4,000 mg of Apap. Some specially compounded products are routinely given to chronic pain patients in doses of up to 180 mg of hydrocodone per day. Tolerance to this drug can increase very rapidly if abused. Because of this, addicts often overdose from taking handfiills of pills, in pursuit of the high they experienced very early on in their hydrocodone use. Symptoms of hydrocodone overdosage include respiratory depression, extreme somnolence, coma, stupor, cold and/or clammy skin, sometimes bradycardia, and hypotension. A severe overdose may involve circulatory collapse, cardiac arrest and/or death.

### C. Background Regarding Naproxen

Naproxen, which is highly water insoluble, i.e., less than 10 mg/ml, has the following chemical structure:

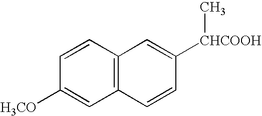

Naproxen is a non-steroidal anti-inflammatory drug (NSAID) often used to relieve the inflammation, swelling, stiffness, and joint pain associated with rheumatoid arthritis, osteoarthritis (the most common form of arthritis), juvenile arthritis, ankylosing spondylitis (spinal arthritis), tendinitis, bursitis, and acute gout. In addition, it is used to treat pain associated with menstrual periods, migraine headaches, and other types of mild to moderate pain.

Naproxen acts by suppressing the production of prostaglandins, which are hormone-like substances that act on local tissues to produce pain and inflammation. Its pharmaceutical forms of delivery include tablets, capsules, and liquids. Delivery characteristics and forms are disclosed in, for example, U.S. Pat. Nos. 3,904,682; 4,009,197; 4,780,320; 4,888,178; 4,919,939; 4,940,588; 4,952,402; 5,200,193; 5,354,556; 5,462,747; and 5,480,650, all of which are specifically incorporated by reference. The synthesis of naproxen is described in U.S. Pat. Nos. 3,904,682 and 4,009,197.

Naproxen is a more potent pain reliever than aspirin, especially for menstrual cramps, toothaches, minor arthritis, and injuries accompanied by inflammation, such as tendinitis. The naproxen sodium salt is specifically indicated in the treatment of various types of acute and very high intensity pain because it induces a rapid and sustained remission. In addition, it is possible to obtain a good analgesic effect with few administrations, due to naproxen's particular pharmacokinetics. Tablet formulations of naproxen were approved for OTC (“over the counter” as compared to prescription) marketing by the U.S. Food and Drug Administration in 1994.

Because of naproxen's low solubility, it is generally formulated for oral administration. However, oral administration of naproxen frequently results in gastrointestinal irritation. All NSAIDs produce gastrointestinal symptoms to some degree upon oral administration. Such symptoms most commonly are constipation, gastric burns, diarrhea, stomatitis, dyspepsia, nausea, vomiting, upper abdominal pain, and heartburn. Oral administration may also lead to an ulcer or bleeding from the stomach or duodenum.

Gastrointestinal irritation resulting from oral administration of an NSAID can be significant. Numerous literature articles detail the severity of gastric irritation caused by NSAID compositions. For example, one report states that between 10,000 and 20,000 people in Canada each year are hospitalized with major gastrointestinal bleeding caused by oral ingestion of NSAIDs, with effects resulting in death for at least 1,000 of these patients. See Marketplace, Oct. 24, 1996. Yet another reference states that gastrointestinal complications of NSAID use may be responsible for over 10,000 deaths each year. See American Family Physician, March 1997.

### D. Background Regarding Nanoparticulate Active Agent Compositions

Nanoparticulate active agent compositions, first described in U.S. Pat. No. 5,145,684 (“the '684 patent”), are particles consisting of a poorly soluble therapeutic or diagnostic agent having adsorbed onto or associated with the surface thereof a non-crosslinked surface stabilizer.

Methods of making nanoparticulate active agent compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. No. 5,399,363 and U.S. Pat. No. 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−) Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract,” 6,592,903 for “Nanoparticulate Dispersions Comprising a Synergistic Combination of a Polymeric Surface Stabilizer and Dioctyl Sodium Sulfosuccinate,” U.S. Pat. No. 6,582,285 for “Apparatus for sanitary wet milling;” U.S. Pat. No. 6,656,504 for “Nanoparticulate Compositions Comprising Amorphous Cyclosporine;” 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” U.S. Pat. No. 6,908,626 for “Compositions having a combination of immediate release and controlled release characteristics;” 6,969,529 for “Nanoparticulate compositions comprising copolymers of vinyl pyrrolidone and vinyl acetate as surface stabilizers;” and 6,976,647 for “System and Method for Milling Materials,” all of which are specifically incorporated by reference. In addition, U.S. Patent Publication No. 20020012675 A1, for “Controlled Release Nanoparticulate Compositions;” U.S. Patent Publication No. 20050276974 for “Nanoparticulate Fibrate Formulations;” U.S. Patent Publication No. 20050238725 for “Nanoparticulate compositions having a peptide as a surface stabilizer;” U.S. Patent Publication No. 20050233001 for “Nanoparticulate megestrol formulations;” U.S. Patent Publication No. 20050147664 for “Compositions comprising antibodies and methods of using the same for targeting nanoparticulate active agent delivery;” U.S. Patent Publication No. 20050063913 for “Novel metaxalone compositions;” U.S. Patent Publication No. 20050042177 for “Novel compositions of sildenafil free base;” U.S. Patent Publication No. 20050031691 for “Gel stabilized nanoparticulate active agent compositions;” U.S. Patent Publication No. 20050019412 for “Novel glipizide compositions;” U.S. Patent Publication No. 20050004049 for “Novel griseofulvin compositions;” U.S. Patent Publication No. 20040258758 for “Nanoparticulate topiramate formulations;” U.S. Patent Publication No. 20040258757 for “Liquid dosage compositions of stable nanoparticulate active agents;” U.S. Patent Publication No. 20040229038 for “Nanoparticulate meloxicam formulations;” U.S. Patent Publication No. 20040208833 for “Novel fluticasone formulations;” U.S. Patent Publication No. 20040195413 for “Compositions and method for milling materials;” U.S. Patent Publication No. 20040156895 for “Solid dosage forms comprising pullulan;” U.S. Patent Publication No. U.S. Patent Publication No. U.S. Patent Publication No. 20040156872 for “Novel nimesulide compositions;” U.S. Patent Publication No. 20040141925 for “Novel triamcinolone compositions;” U.S. Patent Publication No. 20040115134 for “Novel nifedipine compositions;” U.S. Patent Publication No. 20040105889 for “Low viscosity liquid dosage forms;” U.S. Patent Publication No. 20040105778 for “Gamma irradiation of solid nanoparticulate active agents;” U.S. Patent Publication No. 20040101566 for “Novel benzoyl peroxide compositions;” U.S. Patent Publication No. 20040057905 for “Nanoparticulate beclomethasone dipropionate compositions;” U.S. Patent Publication No. 20040033267 for “Nanoparticulate compositions of angiogenesis inhibitors;” U.S. Patent Publication No. 20040033202 for “Nanoparticulate sterol formulations and novel sterol combinations;” U.S. Patent Publication No. 20040018242 for “Nanoparticulate nystatin formulations;” U.S. Patent Publication No. 20040015134 for “Drug delivery systems and methods;” U.S. Patent Publication No. 20030232796 for “Nanoparticulate polycosanol formulations & novel polycosanol combinations;” U.S. Patent Publication No. 20030215502 for “Fast dissolving dosage forms having reduced friability;” U.S. Patent Publication No. 20030185869 for “Nanoparticulate compositions having lysozyme as a surface stabilizer;” U.S. Patent Publication No. 20030181411 for “Nanoparticulate compositions of mitogen-activated protein (MAP) kinase inhibitors;” U.S. Patent Publication No. 20030137067 for “Compositions having a combination of immediate release and controlled release characteristics;” U.S. Patent Publication No. 20030108616 for “Nanoparticulate compositions comprising copolymers of vinyl pyrrolidone and vinyl acetate as surface stabilizers;” U.S. Patent Publication No. 20030095928 for “Nanoparticulate insulin;” U.S. Patent Publication No. 20030087308 for “Method for high through put screening using a small scale mill or microfluidics;” U.S. Patent Publication No. 20030023203 for “Drug delivery systems & methods;” U.S. Patent Publication No. 20020179758 for “System and method for milling materials; and U.S. Patent Publication No. 20010053664 for “Apparatus for sanitary wet milling,” describe nanoparticulate active agent compositions and are specifically incorporated by reference.

In particular, U.S. Pat. No. 5,518,738 for “Nanoparticulate NSAID Formulations;” U.S. Pat. No. 5,552,160 for “Surface Modified NSAID Nanoparticles;” U.S. Pat. No. 5,591,456 for “Milled Naproxen with Hydroxypropyl Cellulose as Dispersion Stabilizer;” U.S. Pat. No. 6,153,225 for “Injectable Formulations of Nanoparticulate Naproxen;” and U.S. Pat. No. 6,165,506 for “New Solid Dose Form of Nanoparticulate Naproxen;” describe nanoparticulate naproxen and are incorporated by reference. None of these patents describe nanoparticulate naproxen in combination with controlled release hydrocodone.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter.” All of the aforementioned patents are hereby incorporated by reference.

The problem with conventional hydrocodone formulations is that they can be habit forming. There is a need in the art for controlled release formulations that may alleviate such side effects. Moreover, there is a need in the art for new combination compositions of controlled release hydrocodone that provide alternatives to the existing combination compositions. The present invention satisfies these needs.

### SUMMARY OF THE INVENTION

It is an object of the present invention to provide a composition comprising nanoparticulate naproxen, or a salt or derivative thereof, in combination with a multiparticulate modified release composition comprising at least two populations of hydrocodone-comprising particles which, upon administration to a patient, exhibit a bimodal or multimodal release profile.

Another object of the invention is to provide a composition comprising nanoparticulate naproxen, or a salt or derivative thereof, in combination with a controlled release hydrocodone composition in which a first portion of the composition, i.e., a hydrocodone or a salt or derivative thereof, is released immediately upon administration and a second portion of the hydrocodone, or a salt or derivative thereof, is released rapidly after an initial delay period in a bimodal manner.

It is another object of the invention to provide a composition comprising nanoparticulate naproxen, or a salt or derivative thereof, in combination with a multiparticulate modified release composition comprising at least two populations of hydrocodone-comprising particles which, upon administration to a patient, exhibits a bimodal or multimodal release profile that results in a plasma profile within therapeutically effective pharmacokinetic parameters.

It is a further object of the invention to provide a composition comprising nanoparticulate naproxen, or a salt or derivative thereof, in combination with a multiparticulate modified release composition comprising at least two populations of hydrocodone-comprising particles which, upon administration to a patient, exhibits a pulsatile release profile, and/or a pulsatile plasma profile.

It is still another object of the invention to provide a composition comprising nanoparticulate naproxen, or a salt or derivative thereof, in combination with a multiparticulate modified release composition comprising at least two populations of hydrocodone-comprising particles which, upon administration to a patient, (1) produces a plasma profile substantially similar to the plasma profile produced by the administration of two or more IR dosage forms given sequentially, and/or (2) substantially mimics the pharmacological and therapeutic effects produced by the administration of two or more IR dosage forms given sequentially.

Conventional frequent dosage regimes in which an immediate release (IR) dosage form is administered at periodic intervals typically gives rise to a pulsatile plasma profile. In this case, a peak in the plasma drug concentration is observed after administration of each IR dose with troughs (regions of low drug concentration) developing between consecutive administration time points. Such dosage regimes (and their resultant pulsatile plasma profiles) have particular pharmacological and therapeutic effects associated with them. For example, the wash out period provided by the fall off of the plasma concentration of the active between peaks has been thought to be a contributing factor in reducing or preventing patient tolerance to various types of drugs.

The present invention further relates to a composition comprising nanoparticulate naproxen, or a salt or derivative thereof, in combination with a controlled release composition comprising hydrocodone, or a salt or derivative thereof, which in operation produced a hydrocodone plasma profile that eliminates the “peaks” and “troughs” produced by the administration of two or more IR dosage forms given sequentially if such a profile is beneficial. This type of profile can be obtained using a controlled release mechanism that allows for “zero-order” delivery. Thus, it is a further object of the invention to provide a composition comprising nanoparticulate naproxen, or a salt or derivative thereof, in combination with a controlled release hydrocodone composition which in operation delivers hydrocodone, or a salt or derivative thereof, in a pulsatile manner or a zero-order manner.

Multiparticulate modified controlled release compositions similar to those disclosed herein are disclosed and claimed in the U.S. Pat. Nos. 6,228,398 and 6,730,325 to Devane et al; both of which are incorporated by reference herein. All of the relevant prior art in this field may also be found therein.

Another object of the invention is to provide a composition comprising nanoparticulate naproxen, or a salt or derivative thereof, in combination with a controlled release composition which substantially reduces or eliminates the development of patient tolerance to hydrocodone, or a salt or derivative thereof.

Another object of the invention is to formulate the dosage in the form of erodable formulations, diffusion controlled formulations, or osmotic controlled formulations.

Another object of the invention is to provide a controlled release composition capable of releasing a hydrocodone or a nanoparticulate naproxen in a bimodal or multi-modal manner in which a first portion of the active is released either immediately or after a delay time to provide a pulse of drug release and one or more additional portions of the hydrocodone or a nanoparticulate naproxen is released, after a respective lag time, to provide additional pulses of drug release during a period of up to twenty-four hours.

It is still a further object of the invention to provide a composition comprising nanoparticulate naproxen, or a salt or derivative thereof, in combination with a multiparticulate modified release composition comprising at least two populations of hydrocodone-comprising particles in which the amount of the one or more active ingredients in the first population of particles is a minor portion of the amount of the one or more active ingredients in the composition, and the amount of the one or more active ingredients in the one or more additional population of particles is a major portion of the amount of the one or more active ingredients in the composition.

It is yet a further object of the invention to provide a solid dosage form comprising the composition comprising nanoparticulate naproxen, or a salt or derivative thereof, in combination with the multiparticulate modified release composition of the present invention. A preferred dosage form of the invention is a solid oral dosage form, although any pharmaceutically acceptable dosage form can be utilized.

Another aspect of the invention is directed to pharmaceutical compositions comprising a composition according to the invention and a pharmaceutically acceptable carrier, as well as any one or more of a number of desired excipients.

One embodiment of the invention encompasses a composition comprising nanoparticulate naproxen, or a salt or derivative thereof, in combination with a multiparticulate modified release composition comprising at least two populations of hydrocodone-comprising particles, wherein the pharmacokinetic profile of the nanoparticulate naproxen, or a salt or derivative thereof, is not affected by the fed or fasted state of a subject ingesting the naproxen composition.

In yet another embodiment, the invention encompasses a composition comprising nanoparticulate naproxen, or a salt or derivative thereof, in combination with a multiparticulate modified release composition comprising at least two populations of hydrocodone-comprising particles, wherein administration of the nanoparticulate naproxen to a subject in a fasted state is bioequivalent to administration of the nanoparticulate naproxen to a subject in a fed state.

In all of the embodiments above, the nanoparticulate naproxen particles have an effective average particle size of less than about 2000 nm, and preferable also comprise at least one surface stabilizer adsorbed on or are associated with the surface of the naproxen particles.

This invention further discloses a method of making the nanoparticulate naproxen compositions. Such a method comprises contacting the naproxen particles with at least one surface stabilizer for a time and under conditions to reduce the effective average particle size of the naproxen particles to less than about 2000 nm.

The present invention is also directed to methods of treatment including but not limited to, the treatment of pain, comprising administering a dosage form comprising a therapeutically effective amount of the composition of the invention to provide bimodal or multimodal release of the hydrocodone comprised therein.

Other objects of the invention include provision of a once daily dosage form of a hydrocodone and a nanoparticulate naproxen which, in operation, produces a plasma profile substantially similar to the plasma profile produced by the administration of two immediate release hydrocodone dosage forms given sequentially and a method for prevention and treatment of pain conditions based on the administration of such a dosage form.

The above objects are realized by a composition comprising a nanoparticulate naproxen, or a salt or derivative thereof, in combination with a controlled release composition having a first component comprising a first population of hydrocodone particles, and a second component or formulation comprising a second population of hydrocodone particles. The hydrocodone-comprising particles of the second component further comprise a modified release constituent comprising a release coating or release matrix material, or both. Following oral delivery, the composition in operation delivers a hydrocodone in a pulsatile or zero order manner. In one embodiment of the invention, the compositions of the invention delivers a hydrocodone in a pulsatile or zero order manner during a period of up to twenty-four hours.

The present invention utilizes controlled release delivery of hydrocodone, or a salt or derivative thereof, from a solid oral dosage formulation to allow dosage less frequently than before, and preferably once-a-day administration, increasing patient convenience and compliance. The mechanism of controlled release would preferably utilize, but not be limited to, erodable formulations, diffusion controlled formulations and osmotic controlled formulations. A portion of the total dose may be released immediately to allow for rapid onset of effect. The invention would be useful in improving compliance and, therefore, therapeutic outcome for all treatments requiring a hydrocodone, including but not limited to, the treatment of pain conditions.

Preferred controlled release formulations are erodable formulations, diffusion controlled formulations and osmotic controlled formulations. According to the invention, a portion of the total dose may be released immediately to allow for rapid onset of effect, with the remaining portion of the total dose released over an extended time period. The invention would be useful in improving compliance and, therefore, therapeutic outcome for all treatments requiring a hydrocodone.

Both the foregoing general description and the following brief description of the figures and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE DRAWINGS

FIG. 1 shows single dose simulations of 10 mg hydrocodone formulations in which 20% of the hydrocodone is contained in the IR component.

FIG. 2 shows single dose simulations of 10 mg hydrocodone formulations in which 20% of the hydrocodone is contained in the IR component.

FIG. 3 shows steady state simulations of 10 mg hydrocodone formulations in which 20% of the hydrocodone is contained in the IR component.

FIG. 4 shows steady state simulations of 10 mg hydrocodone formulations in which 20% of the hydrocodone is contained in the IR component.

FIG. 5 shows single dose simulations of 10 mg hydrocodone formulations in which 50% of the hydrocodone is contained in the IR component.

FIG. 6 shows single dose simulations of 10 mg hydrocodone formulations in which 50% of the hydrocodone is contained in the IR component.

FIG. 7 shows steady state simulations of 10 mg hydrocodone formulations in which 50% of the hydrocodone is contained in the IR component.

FIG. 8 shows steady state simulations of 10 mg hydrocodone formulations in which 50% of the hydrocodone is contained in the IR component.

FIG. 9 shows single dose simulations of 20-160 mg/day hydrocodone formulations (Option 1) in which 20% of the hydrocodone is contained in the IR component.

FIG. 10 shows steady state simulations of 20-160 mg/day hydrocodone formulations (Option 1) in which 20% of the hydrocodone is contained in the IR component.

FIG. 11 shows single dose simulations of 20-80 mg BID hydrocodone formulations (Option 3) in which 20% of the hydrocodone is contained in the IR component.

FIG. 12 shows steady state simulations of 20-80 mg BID hydrocodone formulations (Option 3) in which 20% of the hydrocodone is contained in the IR component.

FIG. 13 shows single dose simulations of 20-160 mg/day hydrocodone formulations (Option 1) in which 50% of the hydrocodone is contained in the IR component.

FIG. 14 shows steady state simulations of 20-160 mg/day hydrocodone formulations (Option 1) in which 50% of the hydrocodone is contained in the IR component.

FIG. 15 shows single dose simulations of 20-160 mg/day hydrocodone formulations (Option 3) in which 50% of the hydrocodone is contained in the IR component.

FIG. 16 shows steady state simulations of 20-160 mg/day hydrocodone formulations (Option 3) in which 50% of the hydrocodone is contained in the IR component.

### DETAILED DESCRIPTION OF THE INVENTION

The compositions of the invention comprise (a) a composition comprising a nanoparticulate naproxen, or a salt or derivative thereof, and at least one surface stabilizer; and (b) an oral controlled release composition of a hydrocodone, or a salt or derivative thereof.

The purpose of the non-controlled release nanoparticulate naproxen composition in combination with the controlled release hydrocodone is at least twofold: (1) to provide increased analgesia via drug synergy; and (2) to limit the intake of hydrocodone by causing unpleasant and often unsafe side effects at higher than prescribed doses.

The present invention provides a method of treating a patient needing pain relief utilizing a composition according to the invention. The method comprises administering a therapeutically effective amount of a dosage form, such as a solid oral dosage form, comprising a nanoparticulate naproxen composition in combination with a controlled release hydrocodone composition, to provide a pulsed or bimodal or zero order delivery of the hydrocodone. Advantages of the present invention include reducing the dosing frequency required by conventional multiple IR dosage regimes while still maintaining the benefits derived from a pulsatile plasma profile or eliminating or minimizing the “peak” to “trough” ratio. This reduced dosing frequency is advantageous in terms of patient compliance to have a formulation which may be administered at reduced frequency. The reduction in dosage frequency made possible by utilizing the present invention would contribute to reducing health care costs by reducing the amount of time spent by health care workers on the administration of drugs.

In one embodiment of the invention, the nanoparticulate naproxen composition, in accordance with standard pharmacokinetic practice, has a bioavailability that is about 100% greater, about 90% greater, about 80% greater, about 70% greater, about 60% greater, about 50% greater, about 40% greater, about 30% greater, about 20% greater, or about 10% greater than a conventional non-nanoparticulate naproxen dosage form.

The compositions of the invention can be administered to a subject via any conventional means including, but not limited to, orally, rectally, ocularly, parenterally (e.g., intravenous, intramuscular, or subcutaneous), intracistem ally, pulmonary, intravaginally, intraperitoneally, locally (e.g., powders, ointments or drops), or as a buccal or nasal spray. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

Solid dosage forms for oral administration include, but are not limited to, capsules, tablets, pills, powders, and granules. In such solid dosage forms, the active agents are admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to hydrocodone and naproxen, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

“Therapeutically effective amount” as used herein with respect to a naproxen and hydrocodone dosage shall mean that dosage that provides the specific pharmacological response for which a hydrocodone or a naproxen is administered in a significant number of subjects in need of such treatment. It is emphasized that “therapeutically effective amount,” administered to a particular subject in a particular instance will not always be effective in treating the conditions described herein, even though such dosage is deemed a “therapeutically effective amount” by those skilled in the art. It is to be further understood that naproxen and hydrocodone dosages are, in particular instances, measured as oral dosages, or with reference to drug levels as measured in blood.

One of ordinary skill will appreciate that effective amounts of a naproxen and a hydrocodone can be determined empirically and can be employed in pure form or, where such forms exist, in pharmaceutically acceptable salt, ester, or prodrug form. Actual dosage levels of a naproxen and a hydrocodone in the compositions of the invention may be varied to obtain an amount of a naproxen and a hydrocodone that is effective to obtain a desired therapeutic response for a particular composition and method of administration. The selected dosage lever therefore depends upon the desired therapeutic effect, the route of administration, the potency of the administered naproxen, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

### I. Controlled Release Hydrocodone

Component of the Compositions of the Invention

### A. Definitions

As used herein, the term “enhancer” refers to a compound which is capable of enhancing the absorption and/or bioavailability of an active ingredient by promoting net transport across the gastrointestinal tract (GIT) in an animal, such as a human. Enhancers include but are not limited to medium chain fatty acids and salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures thereof.

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules or mixture thereof, irrespective of their size, shape or morphology.

The term “modified release” as used herein with respect to the coating or coating material or used in any other context, means release which is not immediate release and is taken to encompass controlled release, sustained release and delayed release.

The term “time delay” as used herein refers to the duration of time between administration of the composition and the release of the hydrocodone from a particular component.

The term “lag time” as used herein refers to the time between delivery of the hydrocodone from one component and the subsequent delivery hydrocodone from another component.

The term “erodable” as used herein refers to formulations which may be worn away, diminished, or deteriorated by the action of substances within the body.

The term “diffusion controlled” as used herein refers to formulations which may spread as the result of their spontaneous movement, for example, from a region of higher to one of lower concentration.

The term “osmotic controlled” as used herein refers to formulations which may spread as the result of their movement through a semipermeable membrane into a solution of higher concentration that tends to equalize the concentrations of the formulation on the two sides of the membrane.

### B. Exemplary Embodiments

The composition according to the invention comprises at least two populations of hydrocodone, or a salt or derivative thereof, comprising particles which have different in vitro dissolution profiles.

The multiparticulate modified release composition and dosage forms made therefrom comprise at least two hydrocodone-comprising components. In one embodiment, the release of the hydrocodone, or a salt or derivative thereof, from the second and subsequent components, if any, is modified such that there is a lag time between the release of hydrocodone, or a salt or derivative thereof, from the first component and each subsequent component. The number of pulses in the release profile arising from such a composition in operation will depend on the number of hydrocodone-comprising components in the composition. For example, a composition comprising two hydrocodone-comprising components will give rise to two pulses in the release profile, and a composition comprising three hydrocodone-comprising components will give rise to up to three pulses in the release profile. In another embodiment, the release of the active ingredients from subsequent components is modified such that the release of hydrocodone from the first component and each subsequent component begins substantially upon administration but over different periods of time and/or at different rates.

For example, a controlled release composition can have a first component comprising a first population of a hydrocodone, or a salt or derivative thereof, and a second component comprising a second population of a hydrocodone, or a salt or derivative thereof. The hydrocodone-comprising particles of the second component are coated with a modified release coating. Alternatively or additionally, the second population of hydrocodone-comprising particles further comprises a modified release matrix material. Following oral delivery, the composition in operation delivers the hydrocodone, or a salt or derivative thereof, in a pulsatile or zero order manner.

In one embodiment of the invention, the controlled release composition comprising hydrocodone, or a salt or derivative thereof, in operation delivers the hydrocodone in a bimodal or pulsatile or zero order manner. Such a composition in operation produces a plasma profile which substantially mimics that obtained by the sequential administration of two IR hydrocodone doses.

The present invention further relates to a controlled release composition comprising a hydrocodone, or a salt or derivative thereof, which in operation produced a plasma profile that eliminates or minimizes the “peaks” and “troughs” produced by the administration of two or more IR dosage forms given sequentially if such a profile is beneficial. This type of profile can be obtained using a controlled release mechanism that allows for “zero-order” delivery.

Any suitable dosage form can be used for the compositions of in the invention. In one embodiment, the invention provides solid oral dosage forms comprising a composition according to the invention.

The time release characteristics for the delivery of the hydrocodone, or a salt or derivative thereof, from each of the components may be varied by modifying the composition of each component, including modifying any of the excipients or coatings which may be present.

In particular, the release of the hydrocodone, or a salt or derivative thereof, may be controlled by changing the composition and/or the amount of the modified release coating on the particles, if such a coating is present. If more than one modified release component is present, the modified release coating for each of these components may be the same or different. Similarly, when modified release is facilitated by the inclusion of a modified release matrix material, release of the hydrocodone, or a salt or derivative thereof, may be controlled by the choice and amount of modified release matrix material utilized. The modified release coating may be present, in each component, in any amount that is sufficient to yield the desired delay time for each particular component. The modified release coating may be preset, in each component, in any amount that is sufficient to yield the desired time lag between components.

The lag time or delay time for the release of the hydrocodone, or a salt or derivative thereof, from each component may also be varied by modifying the composition of each of the components, including modifying any excipients and coatings which may be present. For example, the first component may be an immediate release component wherein the hydrocodone, or a salt or derivative thereof, is released immediately upon administration. Alternatively, the first component may be, for example, a time-delayed immediate release component in which the hydrocodone, or a salt or derivative thereof, is released substantially in its entirety immediately after a time delay. The second component may be, for example, a time-delayed immediate release component as just described or, alternatively, a time-delayed sustained release or extended release component in which the hydrocodone, or a salt or derivative thereof, is released in a controlled fashion over an extended period of time.

It will be understood that suitable hydrocodones also include all pharmaceutically acceptable salts, acids, esters, complexes or other derivatives of hydrocodone, and may be present either in the form of one enantiomer or as a mixture, racemic or otherwise, of enantiomers.

The hydrocodone in each component may be the same or different. In one embodiment, the first component comprises a first hydrocodone, or a salt or derivative thereof, and the second component comprises a second hydrocodone, or a salt or derivative thereof. In another embodiment, two or more hydrocodones may be incorporated into one or more components. Further, a hydrocodone present in one component of the composition may be accompanied by, for example, an enhancer compound or a sensitizer compound in another component of the composition, to modify the bioavailability or therapeutic effect of the hydrocodone.

The amount of the hydrocodone comprised in the composition and in dosage forms made therefrom may be allocated evenly or unevenly across the different particle populations comprising the components of the composition and comprised in the dosage forms made therefrom. In one embodiment, the hydrocodone, or a salt or derivative thereof, comprised in the particles of the first component comprises a minor portion of the total amount of hydrocodone, or a salt or derivative thereof, in the composition or dosage form, and the amount of the hydrocodone, or a salt or derivative thereof, in the other components comprises a major portion of the total amount of hydrocodone, or a salt or derivative thereof, in the composition or dosage form. In one such embodiment comprising two components, about 20% of the total amount of the hydrocodone, or a salt or derivative thereof, is comprised in the particles of the first component, and about 80% of the total amount of the hydrocodone, or a salt or derivative thereof, is comprised in the particles of the second component.

The hydrocodone, or a salt or derivative thereof, is preferably present in the composition and in dosage forms made therefrom in an amount of from about 0.1 to about 1000 mg, from about 1 to about 160 mg, or from about 5 to about 80 mg. Depending at least in part on the particular hydrocodone, or a salt or derivative thereof, that are included in the composition and dosage forms, the hydrocodone, or a salt or derivative thereof, is present in an amount of from about 5 to about 80 mg, about 5 to about 60 mg, about 5 to about 40 mg, about 5 to about 20 mg, about 5 to about 10 mg, about 10 to about 80 mg, about 10 to about 60 mg, about 10 to about 40 mg, about 10 to about 20 mg, about 20 to about 80 mg, about 20 to about 60 mg, about 20 to about 40 mg, about 40 to about 80 mg, about 40 to about 60 mg, or about 60 to about 80 mg.

When the active ingredient is hydrocodone, it is preferably present in the composition and in dosage forms made therefrom in an amount of from about 5 to about 160 mg; more preferably the active ingredient is present in the first component in an amount of from about 10 to about 80 mg.

The profile for the release of the hydrocodone, or a salt or derivative thereof, from each component of the composition may be varied by modifying the composition of each component, including modifying any of the excipients or coatings which may be present. In particular the release of the hydrocodone, or a salt or derivative thereof, may be controlled by the choice and amount of the modified release coating applied to the particles where such a coating is present. If more than one modified release component is present, the modified release coating for each of these components may be the same or different. Similarly, when the modified release is accomplished by means of a modified release matrix material, release of the hydrocodone, or a salt or derivative thereof, may be controlled by the choice and amount of modified release matrix material utilized.

For example, the modified release coating applied to the second population of a hydrocodone, or a salt or derivative thereof, causes a lag time between the release of active from the first population of active hydrocodone-comprising particles and the release of active from the second population of hydrocodone-comprising particles. Similarly, the presence of a modified release matrix material in the second population of hydrocodone-comprising particles causes a lag time between the release of hydrocodone from the first population of hydrocodone comprising particles and the release of hydrocodone, or a salt or derivative thereof, from the second population of hydrocodone-comprising particles. The duration of the lag time may be varied by altering the composition and/or the amount of the modified release coating and/or altering the composition and/or amount of modified release matrix material utilized. Thus, the duration of the lag time can be designed to mimic a desired plasma profile.

In one embodiment, the first component may be an immediate release component wherein the hydrocodone, or a salt or derivative thereof, comprised therein is released substantially immediately upon administration. In another embodiment, the first component may be a delayed release component in which the hydrocodone, or a salt or derivative thereof, is released substantially immediately after a time delay. In either of such embodiments, the second component may be a modified release component in which the hydrocodone, or a salt or derivative thereof, is released over a period of time or substantially immediately after a time delay.

In another embodiment, the controlled release composition comprise an immediate release component and at least one modified release component, the immediate release component comprising a first population of hydrocodone-comprising particles and the modified release components comprising second and subsequent populations of hydrocodone-comprising particles. The second and subsequent modified release components may comprise a controlled release coating. Additionally or alternatively, the second and subsequent modified release components may comprise a modified release matrix material. In operation, administration of such a multi-particulate modified release composition having, for example, a single modified release component results in characteristic pulsatile plasma concentration levels of the hydrocodone in which the immediate release component of the composition gives rise to a first peak in the plasma profile and the modified release component gives rise to a second peak in the plasma profile. Embodiments of the invention comprising more than one modified release component give rise to further peaks in the plasma profile.

As will be appreciated by those skilled in the art, the exact nature of the plasma profile will be influenced by the combination of all of the factors described above. Thus by variation of the composition of each component thereof, including the amount and nature of the hydrocodone, or a salt or derivative thereof, and the modified release coating or modified matrix material, if any, numerous plasma profiles may result therefrom upon administration to a patient. Depending on the release profile of each component, the plasma profile resulting therefrom may be bimodal or multimodal, and may define well separated and clearly defined peaks associated with each component (e.g., when the lag time between immediate release and delayed release components is long) or superimposed peaks associated with each component (e.g., in when the lag time is short). For example, administration of a multiparticulate modified release composition having an immediate release component and a single modified release component can result in a plasma profile in which the immediate release component of the composition gives rise to a first peak in the plasma profile and the modified release component gives rise to a second peak in the plasma profile. Embodiments of the invention comprising more than one modified release component may give rise to further peaks in the plasma profile. Alternatively, administration of a multiparticulate modified release composition having an immediate release component and one or more modified release components can result in a bimodal or multimodal release profile but a plasma profile having a single peak or peaks fewer in number than the number of components contained in the composition.

The plasma profile produced from the administration of a single dosage unit of the present invention is advantageous when it is desirable to deliver two or more portions of hydrocodone, or a salt or derivative thereof, without the need for administration of two or more dosage units. Additionally, in the case of some disorders it is particularly useful to have such a bimodal plasma profile. In embodiments which include drug compounds used for pain management, such as for example hydrocodone, the compositions and dosage forms of the present invention may provide continuous analgesia for up to 24 hours by providing minimum peak to trough fluctuations in plasma levels and reduce or eliminate side effects associated with such drug compounds.

Any coating material which modifies the release of the hydrocodone, or a salt or derivative thereof, in the desired manner may be used in the practice of the present invention. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. ˜5 k-5,000 k), polyvinylpyrrolidone (m. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. −30 k-300 k), polysaccharides such as agar, acacia, karaya, tragacanth, algins and guar, polyacrylamides, Polyox® polyethylene oxides (m. wt. ˜100 k-5,000 k), AquaKeep® acrylate polymers, diesters of polyglucan, crosslinked polyvinyl alcohol and poly N-vinyl-2-pyrrolidone, sodium starch glycolate (e.g., Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g., Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

Excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of a hydrocodone, or a salt or derivative thereof, dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethyl-cellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A multiparticulate modified release composition according to the present invention may be incorporated into any suitable dosage form which facilitates release of the hydrocodone, or a salt or derivative thereof, in a bimodal or multimodal manner. Typically, the dosage form may be a blend of the different populations of a hydrocodone, or a salt or derivative thereof, comprising particles which make up the immediate release and the modified release components, the blend being filled into suitable capsules, such as hard or soft gelatin capsules. Alternatively, the different individual populations of a hydrocodone, or a salt or derivative thereof, comprising particles may be compressed (optionally with additional excipients) into mini-tablets which may be subsequently filled into capsules in the appropriate proportions. Another suitable dosage form is that of a multilayer tablet. In such dosage forms, the first component of the multiparticulate modified release composition may be compressed into one layer with the second component being subsequently added as a second layer of the multilayer tablet. The populations of hydrocodone, or a salt or derivative thereof, comprising particles comprising the composition of the invention may further be included in rapidly dissolving dosage forms such as an effervescent dosage form or a fast-melt dosage form.

In one embodiment, the composition of the invention and the dosage forms made therefrom release the hydrocodone, or a salt or derivative thereof, such that substantially all hydrocodone, or a salt or derivative thereof, comprised in the first component is released prior to release of a hydrocodone, or a salt or derivative thereof, from the second component. For example, when the first component comprises an IR component, release of the hydrocodone, or a salt or derivative thereof, from the second component may be delayed until substantially all the hydrocodone, or a salt or derivative thereof, in the IR component has been released. Release of the hydrocodone, or a salt or derivative thereof, from the second component may be delayed as detailed above by the use of a modified release coating and/or a modified release matrix material.

Because the plasma profile produced by the controlled release hydrocodone composition upon administration can be substantially similar to the plasma profile produced by the administration of two or more IR hydrocodone dosage forms given sequentially, the controlled release composition of the present invention is particularly useful for administering a hydrocodone, or a salt or derivative thereof, for which patient tolerance may be problematical. This controlled release composition is therefore advantageous for reducing or minimizing the development of patient tolerance to the hydrocodone, or a salt or derivative thereof, in the composition.

When it is desirable to minimize patient tolerance by providing a dosage regime which facilitates wash-out of a first dose of hydrocodone, or a salt or derivative thereof, from a patient's system, release of the hydrocodone, or a salt or derivative thereof, from the second component is delayed until substantially all of the hydrocodone, or a salt or derivative thereof, comprised in the first component has been released, and further delayed until at least a portion of the hydrocodone, or a salt or derivative thereof, released from the first component has been cleared from the patient's system. In one embodiment, release of the hydrocodone, or a salt or derivative thereof, from the second component of the composition is substantially, if not completely, delayed for a period of at least about two hours after administration of the composition.

In another embodiment, the composition of the invention and the dosage forms made therefrom release the hydrocodone, or a salt or derivative thereof, such that the hydrocodone, or a salt or derivative thereof, comprised in the first component is released during the release of hydrocodone, or a salt or derivative thereof, from the second component. In one such embodiment, release of the hydrocodone, or a salt or derivative thereof, from the second component of the composition occurs during and beyond the release of the hydrocodone, or a salt or derivative thereof, from the first component.

### C. Other Types of Controlled Release Hydrocodone Compositions

As described herein, the invention includes various types of controlled release systems by which the hydrocodone, or a salt or derivative thereof, may be delivered in a pulsatile or zero order manner. These systems include, but are not limited to: films with the hydrocodone, or a salt or derivative thereof, in a polymer matrix (monolithic devices); the hydrocodone, or a salt or derivative thereof, contained by the polymer (reservoir devices); polymeric colloidal particles or microencapsulates (microparticles, microspheres or nanoparticles) in the form of reservoir and matrix devices; hydrocodone, or a salt or derivative thereof, contained by a polymer containing a hydrophilic and/or leachable additive e.g., a second polymer, surfactant or plasticiser, etc. to give a porous device, or a device in which the hydrocodone, or a salt or derivative thereof, release may be osmotically ‘controlled’ (both reservoir and matrix devices); enteric coatings (ionise and dissolve at a suitable pH); (soluble) polymers with (covalently) attached ‘pendant’ hydrocodone, or a salt or derivative thereof, molecules; devices where release rate is controlled dynamically: e.g., the osmotic pump.

The delivery mechanism of the invention will control the rate of release of the drug. While some mechanisms will release the hydrocodone, or a salt or derivative thereof, at a constant rate (zero order), others will vary as a function of time depending on factors such as changing concentration gradients or additive leaching leading to porosity, etc.

Polymers used in sustained release coatings are necessarily biocompatible, and ideally biodegradable. Examples of both naturally occurring polymers such as Aquacoat® (FMC Corporation, Food & Pharmaceutical Products Division, Philadelphia, USA) (ethylcellulose mechanically spheronised to sub-micron sized, aqueous based, pseudo-latex dispersions), and also synthetic polymers such as the Eudragit® (Rohm Pharma, Weiterstadt.) range of poly(acrylate, methacrylate) copolymers are known in the art.

1. Reservoir Devices

A typical approach to controlled release is to encapsulate or contain the hydrocodone, or a salt or derivative thereof, entirely (e.g., as a core), within a polymer film or coat (i.e., microcapsules or spray/pan coated cores).

The various factors that can affect the diffusion process may readily be applied to reservoir devices (e.g., the effects of additives, polymer functionality {and, hence, sink-solution pH} porosity, film casting conditions, etc.) and, hence, the choice of polymer must be an important consideration in the development of reservoir devices. Modeling the release characteristics of reservoir devices (and monolithic devices) in which the transport of the hydrocodone, or a salt or derivative thereof, is by a solution-diffusion mechanism therefore typically involves a solution to Fick's second law (unsteady-state conditions; concentration dependent flux) for the relevant boundary conditions. When the device contains dissolved hydrocodone, or a salt or derivative thereof, the rate of release decreases exponentially with time as the concentration (activity) of the agent (i.e., the driving force for release) within the device decreases (i.e., first order release). If, however, the hydrocodone, or a salt or derivative thereof, is in a saturated suspension, then the driving force for release is kept constant (zero order) until the device is no longer saturated. Alternatively the release-rate kinetics may be desorption controlled, and a function of the square root of time.

Transport properties of coated tablets, may be enhanced compared to free-polymer films, due to the enclosed nature of the tablet core (permeant) which may enable the internal build-up of an osmotic pressure which will then act to force the permeant out of the tablet.

The effect of deionised water on salt containing tablets coated in poly(ethylene glycol) (PEG)-containing silicone elastomer, and also the effects of water on free films has been investigated. The release of salt from the tablets was found to be a mixture of diffusion through water filled pores, formed by hydration of the coating, and osmotic pumping. KCl transport through films containing just 10% PEG was negligible, despite extensive swelling observed in similar free films, indicating that porosity was necessary for the release of the KCl which then occurred by ‘trans-pore diffusion.’Coated salt tablets, shaped as disks, were found to swell in deionised water and change shape to an oblate spheroid as a result of the build-up of internal hydrostatic pressure: the change in shape providing a means to measure the ‘force’ generated. As might be expected, the osmotic force decreased with increasing levels of PEG content. The lower PEG levels allowed water to be imbibed through the hydrated polymer; whilst the porosity resulting from the coating dissolving at higher levels of PEG content (20 to 40%) allowed the pressure to be relieved by the flow of KCl.

Methods and equations have been developed, which by monitoring (independently) the release of two different salts (e.g., KCl and NaCl) allowed the calculation of the relative magnitudes that both osmotic pumping and trans-pore diffusion contributed to the release of salt from the tablet. At low PEG levels, osmotic flow was increased to a greater extent than was trans-pore diffusion due to the generation of only a low pore number density: at a loading of 20%, both mechanisms contributed approximately equally to the release. The build-up of hydrostatic pressure, however, decreased the osmotic inflow, and osmotic pumping. At higher loadings of PEG, the hydrated film was more porous and less resistant to outflow of salt. Hence, although the osmotic pumping increased (compared to the lower loading), trans-pore diffusion was the dominant release mechanism. An osmotic release mechanism has also been reported for microcapsules containing a water soluble core.

2. Monolithic Devices (Matrix Devices)

Monolithic (matrix) devices are possibly the most common of the devices for controlling the release of drugs. This is possibly because they are relatively easy to fabricate, compared to reservoir devices, and there is not the danger of an accidental high dosage that could result from the rupture of the membrane of a reservoir device. In such a device the hydrocodone, or a salt or derivative thereof, is present as a dispersion within the polymer matrix, and they are typically formed by the compression of a polymer/drug mixture or by dissolution or melting. The dosage release properties of monolithic devices may be dependent upon the solubility of the hydrocodone, or a salt or derivative thereof, in the polymer matrix or, in the case of porous matrices, the solubility in the sink solution within the particle's pore network, and also the tortuosity of the network (to a greater extent than the permeability of the film), dependent on whether the hydrocodone, or a salt or derivative thereof, is dispersed in the polymer or dissolved in the polymer. For low loadings of drug, (0 to 5% W/V) the hydrocodone, or a salt or derivative thereof, will be released by a solution-diffusion mechanism (in the absence of pores). At higher loadings (5 to 10% W/V), the release mechanism will be complicated by the presence of cavities formed near the surface of the device as the hydrocodone, or a salt or derivative thereof, is lost: such cavities fill with fluid from the environment increasing the rate of release of the drug.

It is common to add a plasticiser (e.g., a poly(ethylene glycol)), or surfactant, or adjuvant (i.e., an ingredient which increases effectiveness), to matrix devices (and reservoir devices) as a means to enhance the permeability (although, in contrast, plasticiser may be fugitive, and simply serve to aid film formation and, hence, decrease permeability—a property normally more desirable in polymer paint coatings). It was noted that the leaching of PEG acted to increase the permeability of (ethyl cellulose) films linearly as a function of PEG loading by increasing the porosity, however, the films retained their barrier properties, not permitting the transport of electrolyte. It was deduced that the enhancement of their permeability was as a result of the effective decrease in thickness caused by the PEG leaching. This was evinced from plots of the cumulative permeant flux per unit area as a function of time and film reciprocal thickness at a PEG loading of 50% W/W: plots showing a linear relationship between the rate of permeation and reciprocal film thickness, as expected for a (Fickian) solution-diffusion type transport mechanism in a homogeneous membrane. Extrapolation of the linear regions of the graphs to the time axis gave positive intercepts on the time axis: the magnitude of which decreased towards zero with decreasing film thickness. These changing lag times were attributed to the occurrence of two diffusional flows during the early stages of the experiment (the flow of the ‘drug’ and also the flow of the PEG), and also to the more usual lag time during which the concentration of permeant in the film is building-up. Caffeine, when used as a permeant, showed negative lag times. No explanation of this was forthcoming, but it was noted that caffeine exhibited a low partition coefficient in the system, and that this was also a feature of aniline permeation through polyethylene films which showed a similar negative time lag.

The effects of added surfactants on (hydrophobic) matrix devices has been investigated. It was thought that surfactant may increase the hydrocodone, or a salt or derivative thereof, release rate by three possible mechanisms: (i) increased solubilisation, (ii) improved ‘wettability’ to the dissolution media, and (iii) pore formation as a result of surfactant leaching. For the system studied (Eudragit® RL 100 and RS 100 plasticised by sorbitol, Flurbiprofen as the drug, and a range of surfactants) it was concluded that improved wetting of the tablet led to only a partial improvement in drug release (implying that the release was diffusion, rather than dissolution, controlled), although the effect was greater for Eudragit® RS than Eudragit® RL, whilst the greatest influence on release was by those surfactants that were more soluble due to the formation of ‘disruptions’ in the matrix allowing the dissolution medium access to within the matrix. This is of obvious relevance to a study of latex films which might be suitable for pharmaceutical coatings, due to the ease with which a polymer latex may be prepared with surfactant as opposed to surfactant-free. Differences were found between the two polymers—with only the Eudragit® RS showing interactions between the anionic/cationic surfactant and drug. This was ascribed to the differing levels of quaternary ammonium ions on the polymer.

Composite devices consisting of a polymer/drug matrix coated in a polymer containing no drug also exist. Such a device was constructed from aqueous Eudragit® latices, and was found to give zero order release by diffusion of the drug from the core through the shell. Similarly, a polymer core containing the drug has been produced, but coated this with a shell that was eroded by the gastric fluid. The rate of release of the drug was found to be relatively linear (a function of the rate limiting diffusion process through the shell) and inversely proportional to the shell thickness, whereas the release from the core alone was found to decrease with time.

3. Microspheres

Methods for the preparation of hollow microspheres (‘microballoons’) with the drug dispersed in the sphere's shell, and also highly porous matrix-type microspheres (‘microsponges’) have been described. The microsponges were prepared by dissolving the drug and polymer in ethanol. On addition to water, the ethanol diffused from the emulsion droplets to leave a highly porous particle.

The hollow microspheres were formed by preparing a solution of ethanol/dichloro-methane containing the drug and polymer. On pouring into water, this formed an emulsion containing the dispersed polymer/drug/solvent particles, by a coacervation-type process, from which the ethanol (a good solvent for the polymer) rapidly diffused precipitating polymer at the surface of the droplet to give a hard-shelled particle enclosing the drug, dissolved in the dichloromethane. At this point, a gas phase of dichloromethane was generated within the particle which, after diffusing through the shell, was observed to bubble to the surface of the aqueous phase. The hollow sphere, at reduced pressure, then filled with water, which could be removed by a period of drying. (No drug was found in the water.) A suggested use of the microspheres was as floating drug delivery devices for use in the stomach.

4. Pendent Devices

A means of attaching a range of drugs such as analgesics and antidepressants, etc., by means of an ester linkage to poly(acrylate) ester latex particles prepared by aqueous emulsion polymerization has been developed. These latices when passed through an ion exchange resin such that the polymer end groups were converted to their strong acid form could ‘self-catalyse’ the release of the drug by hydrolysis of the ester link.

Drugs have been attached to polymers, and also monomers have been synthesized with a pendent drug attached. The research group have also prepared their own dosage forms in which the drug is bound to a biocompatible polymer by a labile chemical bond e.g., polyanhydrides prepared from a substituted anhydride (itself prepared by reacting an acid chloride with the drug: methacryloyl chloride and the sodium salt of methoxy benzoic acid) were used to form a matrix with a second polymer (Eudragit® RL) which released the drug on hydrolysis in gastric fluid. The use of polymeric Schiff bases suitable for use as carriers of pharmaceutical amines has also been described.

5. Enteric Films

Enteric coatings consist of pH sensitive polymers. Typically the polymers are carboxylated and interact (swell) very little with water at low pH, whilst at high pH the polymers ionise causing swelling, or dissolving of the polymer. Coatings can therefore be designed to remain intact in the acidic environment of the stomach (protecting either the drug from this environment or the stomach from the drug), but to dissolve in the more alkaline environment of the intestine.

6. Osmotically Controlled Devices

The osmotic pump is similar to a reservoir device but contains an osmotic agent (e.g., the active agent in salt form) which acts to imbibe water from the surrounding medium via a semi-permeable membrane. Such a device, called the ‘elementary osmotic pump’, has been described. Pressure is generated within the device which forces the active agent out of the device via an orifice (of a size designed to minimise solute diffusion, whilst preventing the build-up of a hydrostatic pressure head which has the effect of decreasing the osmotic pressure and changing the dimensions {volume} of the device). Whilst the internal volume of the device remains constant, and there is an excess of solid (saturated solution) in the device, then the release rate remains constant delivering a volume equal to the volume of solvent uptake.

7. Electrically Stimulated Release Devices

Monolithic devices have been prepared using polyelectrolyte gels which swelled when, for example, an external electrical stimulus was applied, causing a change in pH. The release could be modulated, by the current, giving a pulsatile release profile.

8. Hydrogels

Hydrogels find a use in a number of biomedical applications, in addition to their use in drug matrices (e.g., soft contact lenses, and various ‘soft’ implants, etc.).

### II. Nanoparticulate Naproxen Component of the Compositions of the Invention

The compositions of the invention comprise a nanoparticulate naproxen composition. The nanoparticulate naproxen composition comprises particles of naproxen having an effective average particle size of less than about 2000 nm and preferably at least one surface stabilizer adsorbed on or associated with the surface of the drug.

Advantages of the nanoparticulate naproxen compositions of the invention as compared to conventional, non-nanoparticulate or solubilized dosage forms of naproxen include, but are not limited to: (1) smaller tablet or other solid dosage form size; (2) smaller doses of drug required to obtain the same pharmacological effect; (3) increased bioavailability; (4) substantially similar pharmacokinetic profiles of the naproxen compositions when administered in the fed versus the fasted state; (5) bioequivalency of the naproxen compositions when administered in the fed versus the fasted state; (6) an increased rate of dissolution for the naproxen compositions; and (7) the naproxen compositions can be used in conjunction with other active agents useful in the prevention and treatment of infective conditions.

The present invention also includes nanoparticulate naproxen compositions together with one or more non-toxic physiologically acceptable carriers, adjuvants, or vehicles, collectively referred to as carriers. The compositions can be formulated for parental injection (e.g., intravenous, intramuscular, or subcutaneous), oral administration in solid, liquid, or aerosol form, vaginal, nasal, rectal, ocular, local (powders, ointments, or drops), buccal, intracisternal, intraperitoneal, or topical administrations, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

The present invention is described herein using several definitions, as set forth below and throughout the application.

The term “effective average particle size,” as used herein, means that at least about 50% of the nanoparticulate naproxen particles have a size of less than about 2000 nm, by weight or by other suitable measurement technique (e.g., such as by volume, number, etc.), when measured by, for example, sedimentation flow fractionation, photon correlation spectroscopy, light scattering, disk centrifugation, and other techniques known to those of skill in the art.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein with reference to stable naproxen particles, “stable” means that the particles do not appreciably flocculate or agglomerate due to interparticle attractive forces or otherwise increase in particle size. “Stable” connotes, but is not limited to one or more of the following parameters: (1) the particles do not appreciably flocculate or agglomerate due to interparticle attractive forces or otherwise significantly increase in particle size over time; (2) the physical structure of the particles is not altered over time, such as by conversion from an amorphous phase to a crystalline phase; (3) the particles are chemically stable; and/or (4) where the naproxen or a salt or derivative thereof has not been subject to a heating step at or above the melting point of the naproxen particles in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to drugs having a solubility in water of less than about 30 mg/ml, less than about 20 mg/ml, less than about 10 mg/ml, or less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

A. Preferred Characteristics of the Nanoparticulate Naproxen Compositions of the Invention

1. Increased Bioavailability

The nanoparticulate naproxen formulations of the invention exhibit increased bioavailability, and require smaller doses as compared to prior conventional, non-nanoparticulate naproxen formulations.

2. Improved Pharmacokinetic Profiles

The invention also provides nanoparticulate naproxen, or a salt or derivative thereof, compositions having a desirable pharmacokinetic profile when administered to mammalian subjects. The desirable pharmacokinetic profile of the compositions comprising naproxen includes but is not limited to: (1) a Cₘₐₓ for naproxen, when assayed in the plasma of a mammalian subject following administration, that is preferably greater than the Cₘₐₓ for a non-nanoparticulate formulation of the same naproxen, administered at the same dosage; and/or (2) an AUC for naproxen, when assayed in the plasma of a mammalian subject following administration, that is preferably greater than the AUC for a non-nanoparticulate formulation of the same naproxen, administered at the same dosage; and/or (3) a Tₘₐₓ for naproxen, when assayed in the plasma of a mammalian subject following administration, that is preferably less than the Tₘₐₓ for a non-nanoparticulate formulation of the same naproxen, administered at the same dosage. The desirable pharmacokinetic profile, as used herein, is the pharmacokinetic profile measured after the initial dose of naproxen or a salt or derivative thereof.

In one embodiment, a composition comprising a nanoparticulate naproxen exhibits in comparative pharmacokinetic testing with a non-nanoparticulate formulation of the same naproxen, administered at the same dosage, a Tₘₐₓ not greater than about 90%, not greater than about 80%, not greater than about 70%, not greater than about 60%, not greater than about 50%, not greater than about 40%, not greater than about 30%, not greater than about 25%, not greater than about 20%, not greater than about 15%, not greater than about 10%, or not greater than about 5% of the Tₘₐₓ exhibited by the non-nanoparticulate naproxen formulation.

In another embodiment, the composition comprising a nanoparticulate naproxen exhibits in comparative pharmacokinetic testing with a non-nanoparticulate formulation of the same naproxen, administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate naproxen formulation.

In yet another embodiment, the composition comprising a nanoparticulate naproxen exhibits in comparative pharmacokinetic testing with a non-nanoparticulate formulation of the same naproxen, administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate naproxen formulation.

In one embodiment of the invention, the Tₘₐₓ of naproxen, when assayed in the plasma of the mammalian subject, is less than about 6 to about 8 hours. In other embodiments of the invention, the Tₘₐₓ of naproxen is less than about 6 hours, less than about 5 hours, less than about 4 hours, less than about 3 hours, less than about 2 hours, less than about 1 hour, or less than about 30 minutes after administration.

The desirable pharmacokinetic profile, as used herein, is the pharmacokinetic profile measured after the initial dose of naproxen or a salt or derivative thereof. The compositions can be formulated in any way as described herein and as known to those of skill in the art.

3. The Pharmacokinetic Profiles of the Naproxen Compositions of the Invention are not Affected by the Fed or Fasted State of the Subject Ingesting the Compositions

The invention encompasses naproxen compositions wherein the pharmacokinetic profile of naproxen is not substantially affected by the fed or fasted state of a subject ingesting the composition. This means that there is no substantial difference in the quantity of drug absorbed or the rate of drug absorption when the nanoparticulate naproxen compositions are administered in the fed versus the fasted state.

Benefits of a dosage form, which substantially eliminates the effect of food, include an increase in subject convenience, thereby increasing subject compliance, as the subject does not need to ensure that they are taking a dose either with or without food. This is significant, as with poor subject compliance an increase in the medical condition for which the drug is being prescribed may be observed.

4. Bioequivalency of Naproxen Compositions of the Invention when Administered in the Fed Versus the Fasted State

The invention also encompasses provides a nanoparticulate naproxen composition in which administration of the composition to a subject in a fasted state is bioequivalent to administration of the composition to a subject in a fed state.

The difference in absorption (AUC) or Cₘₐₓ of the nanoparticulate naproxen compositions of the invention, when administered in the fed versus the fasted state, preferably is less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In one embodiment of the invention, the invention encompasses compositions comprising a nanoparticulate naproxen, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

5. Dissolution Profiles of the Naproxen Compositions of the Invention

The nanoparticulate naproxen compositions of the invention are proposed to have unexpectedly dramatic dissolution profiles. Rapid dissolution of an administered active agent is preferable, as faster dissolution generally leads to faster onset of action and greater bioavailability. To improve the dissolution profile and bioavailability of the naproxen, it would be useful to increase the drug's dissolution so that it could attain a level close to 100%.

The naproxen compositions of the invention preferably have a dissolution profile in which within about 5 minutes at least about 20% of the composition is dissolved. In other embodiments of the invention, at least about 30% or at least about 40% of naproxen composition is dissolved within about 5 minutes. In yet other embodiments of the invention, preferably at least about 40%, at least about 50%, at least about 60%, at least about 70%, or at least about 80% of the naproxen composition is dissolved within about 10 minutes. Finally, in another embodiment of the invention, preferably at least about 70%, at least about 80%, at least about 90%, or at least about 100% of the naproxen composition is dissolved within about 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

6. Redispersibility Profiles of the Naproxen Compositions of the Invention

An additional feature of the naproxen compositions of the invention is that the compositions redisperse such that the effective average particle size of the redispersed naproxen particles is less than about 2 microns. This is significant, as if upon administration the naproxen compositions of the invention did not redisperse to a substantially nanoparticulate particle size, then the dosage form may lose the benefits afforded by formulating the naproxen into a nanoparticulate particle size.

This is because nanoparticulate active agent compositions benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall well below that observed with the liquid dispersion form of the nanoparticulate active agent.

Moreover, the nanoparticulate naproxen or a salt or derivative thereof compositions of the invention exhibit dramatic redispersion of the nanoparticulate naproxen particles upon administration to a mammal, such as a human or animal, as demonstrated by reconstitution/redispersion in a biorelevant aqueous media such that the effective average particle size of the redispersed naproxen particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed naproxen particles of the invention (redispersed in an aqueous, biorelevant, or any other suitable media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

B. Nanoparticulate Naproxen Compositions

The invention provides compositions comprising naproxen particles and at least one surface stabilizer. The surface stabilizers preferably are adsorbed on, or associated with, the surface of the naproxen particles. Surface stabilizers especially useful herein preferably physically adhere on, or associate with, the surface of the nanoparticulate naproxen particles, but do not chemically react with the naproxen particles or itself. Individually adsorbed molecules of the surface stabilizer are essentially free of intermolecular cross-linkages.

The invention also includes naproxen compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracistemal, intraperitoneal, or topical administration, and the like.

1. Naproxen Particles

The compositions of the invention comprise particles of naproxen or a salt or derivative thereof. The particles can be in a crystalline phase, semi-crystalline phase, amorphous phase, semi-amorphous phase, or a combination thereof.

2. Surface Stabilizers

Combinations of more than one surface stabilizers can be used in the invention. Useful surface stabilizers which can be employed in the invention include, but are not limited to, known organic and inorganic pharmaceutical excipients. Such excipients include various polymers, low molecular weight oligomers, natural products, and surfactants. Exemplary surface stabilizers include nonionic, ionic anionic, cationic, and zwitterionic surfactants or compounds.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68@ and F108®, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda, Inc.); p-isononylphenoxypoly-(glycidol), also known as Olin-OG® or Surfactant 10-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅-dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, polydiallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquarternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™)

Suitable lubricants, including agents that act on the flowability of the powder to be compressed, are colloidal silicon dioxide, such as Aerosil®200, talc, stearic acid, magnesium stearate, calcium stearate, and silica gel.

Examples of sweeteners are any natural or artificial sweetener, such as sucrose, xylitol, sodium saccharin, cyclamate, aspartame, and acesulfame. Examples of flavoring agents are Magnasweet (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Nanoparticulate Naproxen Particle Size

The compositions of the invention comprise naproxen particles which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the naproxen particles have a particle size of less than the effective average, by weight or by another suitable measurement technique (e.g., volume, number, etc.), i.e., less than about 2000 nm, 1900 nm, 1800 nm, etc., when measured by the above-noted techniques. In other embodiments of the invention, at least about 60%, at least about 70%, at least about 80%, at least about 90%, at least about 95%, or at least about 99% of the naproxen particles have a particle size of less than the effective average, i.e., less than about 2000 nm, 1900 nm, 1800 nm, 1700 nm, etc.

In the present invention, the value for D50 of a nanoparticulate naproxen composition is the particle size below which 50% of the naproxen particles fall, by weight. Similarly, D90 is the particle size below which 90% of the naproxen particles fall, by weight.

5. Concentration of Naproxen and Surface Stabilizers

The relative amounts of naproxen and one or more surface stabilizers can vary widely. The optimal amount of the individual components can depend, for example, upon the particular naproxen selected, the hydrophilic lipophilic balance (HLB), melting point, and the surface tension of water solutions of the stabilizer, etc.

The concentration of naproxen can vary from about 99.5% to about 0.001%, from about 95% to about 0.1%, or from about 90% to about 0.5%, by weight, based on the total combined dry weight of naproxen and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of naproxen and at least one surface stabilizer, not including other excipients.

C. Methods of Making Nanoparticulate Naproxen Compositions

The nanoparticulate naproxen compositions can be made using, for example, milling, homogenization, precipitation, freezing, or template emulsion techniques. Exemplary methods of making nanoparticulate compositions are described in the '684 patent. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate naproxen compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

1. Milling to Obtain Nanoparticulate Naproxen Dispersions

Milling a naproxen to obtain a nanoparticulate dispersion comprises dispersing the naproxen particles in a liquid dispersion medium in which the naproxen is poorly soluble, followed by applying mechanical means in the presence of grinding media to reduce the particle size of the naproxen to the desired effective average particle size. The dispersion medium can be, for example, water, safflower oil, ethanol, t-butanol, glycerin, polyethylene glycol (PEG), hexane, or glycol. A preferred dispersion medium is water.

The naproxen particles can be reduced in size in the presence of at least one surface stabilizer. Alternatively, naproxen particles can be contacted with one or more surface stabilizers after attrition. Other compounds, such as a diluent, can be added to the naproxen/surface stabilizer composition during the size reduction process. Dispersions can be manufactured continuously or in a batch mode.

2. Precipitation to Obtain Nanoparticulate Naproxen Compositions

Another method of forming the desired nanoparticulate naproxen composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the naproxen in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Naproxen Compositions

Exemplary homogenization methods of preparing active agent nanoparticulate compositions are described in U.S. Pat. No. 5,510,118, for “Process of Preparing Therapeutic Compositions Containing Nanoparticles.” Such a method comprises dispersing particles of a naproxen in a liquid dispersion medium, followed by subjecting the dispersion to homogenization to reduce the particle size of a naproxen to the desired effective average particle size. The naproxen particles can be reduced in size in the presence of at least one surface stabilizer. Alternatively, the naproxen particles can be contacted with one or more surface stabilizers either before or after attrition. Other compounds, such as a diluent, can be added to the naproxen/surface stabilizer composition, either before, during, or after the size reduction process. Dispersions can be manufactured continuously or in a batch mode.

4. Cryogenic Methodologies to Obtain Nanoparticulate Naproxen Compositions

Another method of forming the desired nanoparticulate naproxen composition is by spray freezing into liquid (SFL). This technology comprises an organic or organoaqueous solution of naproxen with stabilizers, which is injected into a cryogenic liquid, such as liquid nitrogen. The droplets of the naproxen solution freeze at a rate sufficient to minimize crystallization and particle growth, thus formulating nanostructured naproxen particles. Depending on the choice of solvent system and processing conditions, the nanoparticulate naproxen particles can have varying particle morphology. In the isolation step, the nitrogen and solvent are removed under conditions that avoid agglomeration or ripening of the naproxen particles.

As a complementary technology to SFL, ultra rapid freezing (URF) may also be used to created equivalent nanostructured naproxen particles with greatly enhanced surface area. URF comprises an organic or organoaqueous solution of naproxen with stabilizers onto a cryogenic substrate.

5. Emulsion Methodologies to Obtain Nanoparticulate Naproxen Compositions

Another method of forming the desired nanoparticulate naproxen composition is by template emulsion. Template emulsion creates nanostructured naproxen particles with controlled particle size distribution and rapid dissolution performance. The method comprises an oil-in-water emulsion that is prepared, then swelled with a non-aqueous solution comprising the naproxen and stabilizers. The particle size distribution of the naproxen particles is a direct result of the size of the emulsion droplets prior to loading with the naproxen a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured naproxen particles are recovered. Various naproxen particles morphologies can be achieved by appropriate control of processing conditions.

In the following examples all percentages are weight by weight unless otherwise stated. The term “purified water” as used throughout the Examples refers to water that has been purified by passing it through a water filtration system. It is to be understood that the examples are for illustrative purposes only, and should not be interpreted as restricting the spirit and scope of the invention, as defined by the scope of the claims that follow. All references identified herein, including U.S. patents, are hereby expressly incorporated by reference.

### EXAMPLE 1

The purpose of this example is to describe preparation of a multiparticulate modified release composition comprising a hydrocodone that can be used in the combination compositions of the invention.

Multiparticulate modified release hydrocodone compositions according to the present invention having an immediate release component and a modified release component having a modified release coating are prepared according to the formulations shown in Tables 1 and 2.

TABLE 1
Immediate Release Component Hydrocodone Solutions

| | Amount, % (w/w) | | | | | |
|---|---|---|---|---|---|---|
| Ingredient | (i) | (ii) | (iii) | (iv) | (v) | (vi) |
| Hydrocodone Bitartrate | 6.0 | 6.0 | 6.0 | 6.0 | 6.0 | 6.0 |
| HPMC 2910 | 1.0 | 2.0 | 2.0 | — | — | 1.5 |
| Polyethylene Glycol 6000 | — | — | — | 0.5 | — | — |
| Povidone K30 | — | — | — | — | 5.0 | — |
| Fumaric Acid | — | 6.0 | — | — | — | — |
| Citric Acid | — | — | 6.0 | — | — | — |
| Silicon Dioxide | 1.5 | 1.0 | 1.0 | — | — | 2.0 |
| Talc | 1.5 | — | — | — | — | — |
| Purified Water | 90.0 | 85.0 | 85.0 | 93.5 | 89.0 | 90.5 |

TABLE 2
Modified Release Component Hydrocodone Solutions

| | Amount, % (w/w) | | | | | | |
|---|---|---|---|---|---|---|---|
| Ingredient | (i) | (ii) | (iii) | (iv) | (iv) | (vi) | (vii) |
| Eudragit RS 100 | 4.1 | 4.9 | 5.5 | 4.4 | — | 5.5 | 7.5 |
| Eudragit RL 100 | — | 0.5 | — | 1.1 | — | — | — |
| Eudragit L 100 | 1.4 | — | — | — | — | — | — |
| Ethocel | — | — | — | — | 3.0 | — | — |
| Triethyl Citrate | 1.5 | 1.6 | — | 1.1 | — | — | 1.5 |
| Dibutyl Sebacate | — | — | — | — | 0.6 | 1.0 | — |
| Silicon Dioxide | 1.0 | 1.0 | 1.0 | — | 2.0 | 1.0 | — |
| Talc | 2.5 | 2.5 | 1.0 | 2.8 | — | 1.0 | 2.5 |
| Acetone | 34.0 | 34.0 | 15.0 | 35.6 | — | 14.0 | 33.5 |
| Isopropyl Alcohol | 50.0 | 50.0 | 72.5 | 50.0 | 94.4 | 72.5 | 50.0 |
| Purified Water | 5.5 | 5.5 | 5.0 | 5.0 | — | 5.0 | 5.0 |

In these exemplary hydrocodone formulations, the sugar spheres (30/35 mesh) are provided as inert cores that act as a carrier for the active ingredient and other excipients present in the formulation. The quality and size selected reflect the requirement to produce multiparticulates with a mean diameter in the size range 0.5-0.6 mm to facilitate the subsequent coating and encapsulation process. Hydroxypropylmethylcellulose (2910) (Methocal E6 Premium LV) is used to prepare the immediate-release coating solution that is coated onto the sugar spheres to produce the IR beads and acts as a binding agent. Silicon Dioxide (Syloid 244FP) is an anti-adherent that is used in the preparation of the IR coating solution (Table 1) and the modified release coating suspension (Table 2).

Ammonio methyacrylate copolymer Type B (Eudragit RS 100) is a rate-controlling polymer that imparts the controlled release properties to the formulation and exhibits pH independent release properties. Talc (Altaic 200) is used as an anti-adherent in the modified-release coating process to manufacture the modified release beads. Acetone and isopropyl alcohol are the two solvents in which the rate-controlling polymer is dissolved to produce the coating suspension that is applied to the IR beads to form the modified release beads. The resultant coating suspension is applied to the IR beads to form the modified release beads. Modified release beads are dried in an oven for 10-20 hours at 40-500C/30-60% RH to remove residual solvents and to obtain a moisture content of about 3-6%. Suitable processing procedures are further detailed in U.S. Pat. No. 6,066,339 which is incorporated herein by reference in its entirety.

Table 3 shows the dissolution profiles for two multiparticulate modified release formulations prepared in accordance with Tables 1 and 2. These results indicate that about 20% of the hydrocodone was released in the first hour and about 80% of the hydrocodone was released over a period of about 11 hours.

TABLE 3
Dissolution Data for Compositions Containing an
IR Component and a Modified Release Component

Formulation

| Time (hr) | Fumaric Acid | Non-Fumaric Acid |
|---|---|---|
| 0 | 0 | 0 |
| 1 | 22 | 26 |
| 2 | 33 | 31 |
| 4 | 54 | 54 |
| 6 | 68 | 64 |
| 8 | 77 | 73 |
| 12 | 93 | 86 |

### In Vivo Study

A randomized, single-dose, parallel-group, placebo-controlled, active-comparator study was performed to evaluate the safety, efficacy, and PK of hydrocodone formulations in subjects immediately following bunionectomy study. The study treatments were 10, 20, 30, 40 mg of hydrocodone bitartarate, matching active comparator (10 mg hydrocodone/APAP) or matching placebo. During the 24-hour confinement periods, blood was collected at baseline and at up to 17 additional time points, from 115 subjects (approx. 17 to 21 subjects per group), to determine the concentrations in plasma of hydrocodone. The following PK parameters were calculated and are presented in Tables 4-6.

TABLE 4

| | | HC ER 10 mg | HC ER 20 mg | HC ER 30 mg | HC ER 40 mg | HC/APAP | Placebo |
|---|---|---|---|---|---|---|---|
| Parameter | Statistics | N = 21 | N = 19 | N = 19 | N = 17 | N = 18 | N = 21 |
| Cmax (ng/mL) | n | 21 | 19 | 19 | 17 | 18 | 21 |
| | Mean | 8.9 | 17.9 | 31.7 | 37.5 | 19.5 | 0.1 |
| | Std. Dev. | 2.11 | 5.85 | 8.50 | 8.82 | 8.69 | 0.17 |
| | Median | 9.1 | 16.3 | 30.1 | 34.1 | 20.2 | 0.0 |
| | Min/Max | 5/15 | 10/27 | 16/46 | 28/62 | 9/45 | 0/1 |
| Tmax (hr) | n | 21 | 19 | 19 | 17 | 18 | 3 |
| | Mean | 6.3 | 6.0 | 6.3 | 6.1 | 2.7 | 8.2 |
| | Std. Dev. | 1.46 | 1.80 | 1.88 | 1.62 | 1.66 | 13.70 |
| | Median | 6.1 | 5.2 | 6.1 | 6.0 | 2.1 | 0.6 |
| | Min/Max | 4/9 | 4/12 | 4/10 | 4/10 | 1/7 | 0/24 |
| kel (1/hr) | n | 21 | 19 | 19 | 17 | 18 | NC (a) |
| | Mean | 0.090 | 0.095 | 0.086 | 0.079 | 0.138 | NC |
| | Std. Dev. | 0.0276 | 0.0289 | 0.0229 | 0.0211 | 0.0297 | NC |
| | Median | 0.092 | 0.089 | 0.083 | 0.079 | 0.147 | NC |
| | Min/Max | 0.02/0.13 | 0.05/0.16 | 0.05/0.13 | 0.05/0.13 | 0.06/0.18 | NC |

(a) NC = Not Calculated

TABLE 5

| | | HC ER 10 mg | HC ER 20 mg | HC ER 30 mg | HC ER 40 mg | HC/APAP | Placebo |
|---|---|---|---|---|---|---|---|
| Parameter | Statistics | N = 21 | N = 19 | N = 19 | N = 17 | N = 18 | N = 21 |
| t½ (hr) | n | 21 | 19 | 19 | 17 | 18 | NC |
| | Mean | 9.5 | 7.9 | 8.6 | 9.4 | 5.3 | NC |
| | Std. Dev. | 8.25 | 2.44 | 2.32 | 2.40 | 1.64 | NC |
| | Median | 7.6 | 7.8 | 8.4 | 8.8 | 4.7 | NC |
| | Min/Max | 5/45 | 4/15 | 5/13 | 5/14 | 4/11 | NC |
| AUCIast (ng * hr/mL) | n | 21 | 19 | 19 | 17 | 18 | 21 |
| | Mean | 109.0 | 212.9 | 392.5 | 464.6 | 131.2 | 0.1 |
| | Std. Dev. | 27.25 | 73.19 | 117.74 | 124.01 | 36.80 | 0.19 |
| | Median | 104.2 | 196.2 | 367.0 | 471.0 | 129.9 | 0.0 |
| | Min/Max | 73/179 | 130/377 | 177/671 | 321/712 | 80/162 | 011 |
| AUCinf (ng * hr/mL) | n | 21 | 19 | 19 | 17 | 18 | NC |
| | Mean | 136.9 | 255.6 | 480.7 | 596.2 | 137.6 | NC |
| | Std. Dev. | 39.48 | 88.66 | 138.70 | 172.73 | 39.99 | NC |
| | Median | 128.1 | 252.7 | 459.5 | 578.0 | 135.4 | NC |
| | Min/Max | 80/217 | 151/468 | 226/756 | 375/992 | 83/189 | NC |

TABLE 6

| | | HC ER 10 mg | HC ER 20 mg | HC ER 30 mg | HC ER 40 mg | HC/APAP | Placebo |
|---|---|---|---|---|---|---|---|
| Ratio Using AUClast | Statics | N = 21 | N = 19 | N = 19 | N = 17 | N = 18 | N = 21 |
| Hydromorphone/ | n | 21 | 19 | 19 | 17 | 18 | 3 |
| hydrocodone | Mean | 0.000 | 0.001 | 0.002 | 0.003 | 0.001 | 0.000 |
| | Std. Dev. | 0.0009 | 0.0038 | 0.0027 | 0.0050 | 0.0012 | 0.0000 |
| | Median | 0.000 | 0.000 | 0.001 | 0.002 | 0.000 | 0.000 |
| | Min/Max | 0.00/0.00 | 0.00/0.02 | 0.00/0.01 | 0.00/0.02 | 0.00/0.00 | 0.00/0.00 |
| Norhydrocodone/ | n | 21 | 19 | 19 | 17 | 18 | 3 |
| Hydrocodone | Mean | 0.366 | 0.360 | 0.327 | 0.362 | 0.448 | 0.000 |
| | Std. Dev. | 0.1189 | 0.1215 | 0.1243 | 0.1310 | 0.2144 | 0.0000 |
| | Median | 0.368 | 0.324 | 0.297 | 0.334 | 0.400 | 0.000 |
| | Min/Max | 0.11/0.61 | 0.17/0.58 | 0.20/0.76 | 0.23/0.74 | 0.22/0.84 | 0.00/0.00 |

### Hydrocodone Simulations

Studies of hydrocodone formulations of the present invention were conducted to simulate the profiles associated with twice-daily administration hydrocodone for both single dose and steady state. The target doses were 10, 20, 40 and 80 mg, and the targeted minimum concentration was 5-10 ng/ml. The formulations of the study were two-component dosage forms comprising an immediate release component and a modified release component in which the hydrocodone was allocated evenly (50/50) or unevenly (20/80) across the two components. Non-compartmental parameters were used to find estimates of the unit input response and a one-compartment model was assumed for all simulations.

Non-compartmental parameters following a 10 mg oral dose of hydrocodone administered to five adult males are reported as shown in Table 7 below.

TABLE 7
Non-Compartmental Parameters

| | | |
|---|---|---|
| Cmax | 23.6 ± 5.2 | ng/ml |
| Tmax | 1.3 ± 0.3 | hours |
| ^{T}half | 3.8 ± 0.3 | hours |

K10 and V/f were estimated to be 0.18 and 334.29 L respectively. For the absorption rate constant k01, several profiles were simulated using different estimates of k01. The secondary parameters estimates were compared to identify an appropriate ka as set forth in Table 8 below.

TABLE 8
Comparison of Absorption Rate Constant (ka).

| | | |
|---|---|---|
| ka = 1 | AUC | 166.19 |
| ka = 1 | KO1-HL | 0.69 |
| ka = 1 | K10-HL | 3.85 |
| ka = 1 | CL/F | 60.17 |
| ka = 1 | Tmax | 2.09 |
| ka = 1 | Cmax | 20.53 |
| ka = 2 | AUC | 166.19 |
| ka = 2 | KO1-HL | 0.35 |
| ka = 2 | K10-HL | 3.85 |
| ka = 2 | CL/F | 60.17 |
| ka = 2 | Tmax | 1.32 |
| ka = 2 | Cmax | 23.57 |
| ka = 6 | AUC | 166.19 |
| ka = 6 | KO1-HL | 0.12 |
| ka = 6 | K10-HL | 3.85 |
| ka = 6 | CL/F | 60.17 |
| ka = 6 | Tmax | 0.60 |
| ka⁼6 | Cmax | 26.84 |

ka=2 appeared to be the best estimate of the absorption rate of the instant release hydrocodone given that the maximum concentration observed and the time to maximum concentration were comparable to previous data set forth above.

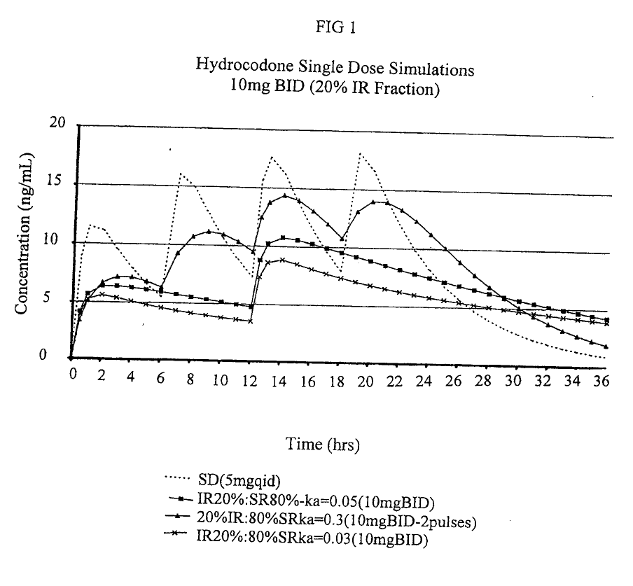

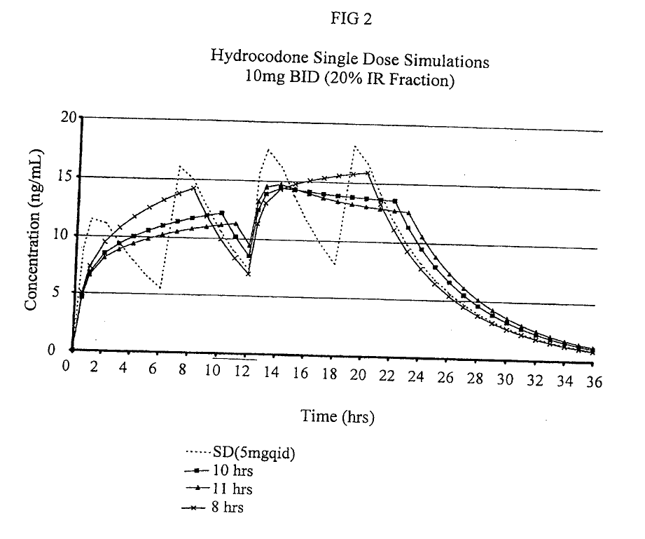

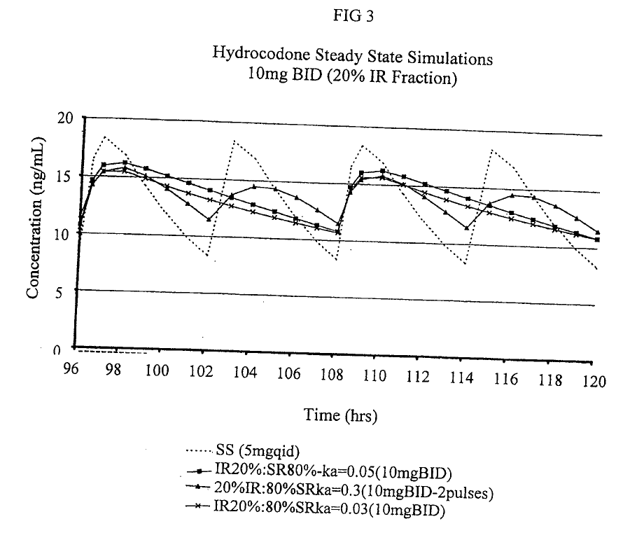

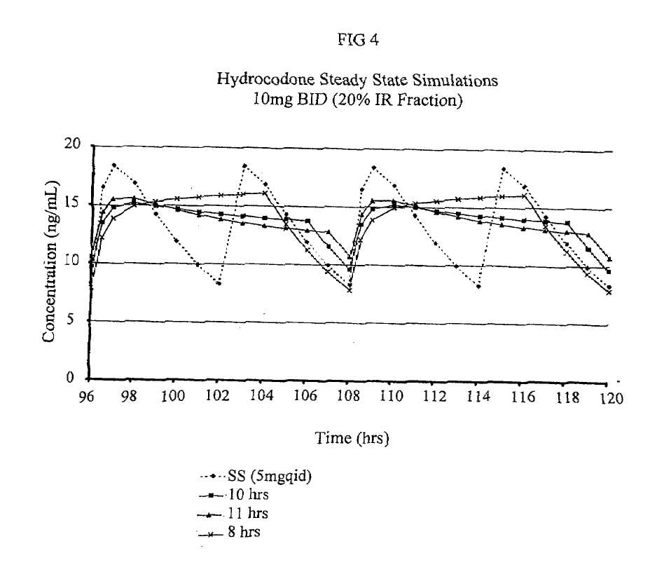

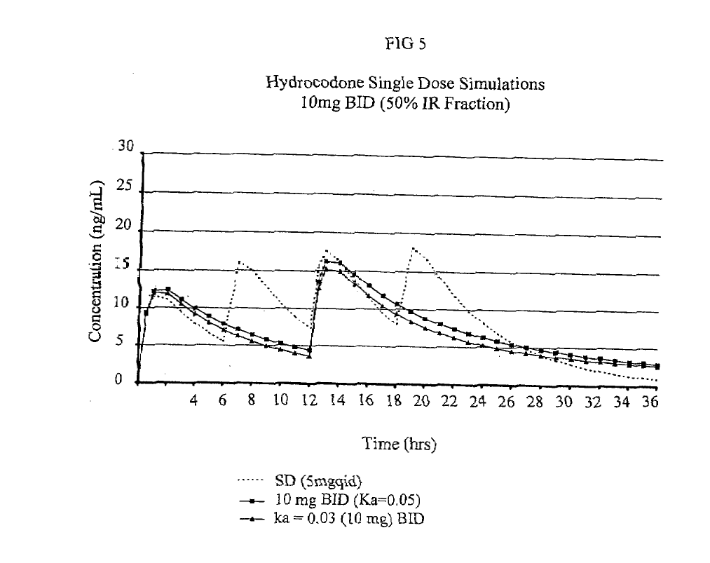

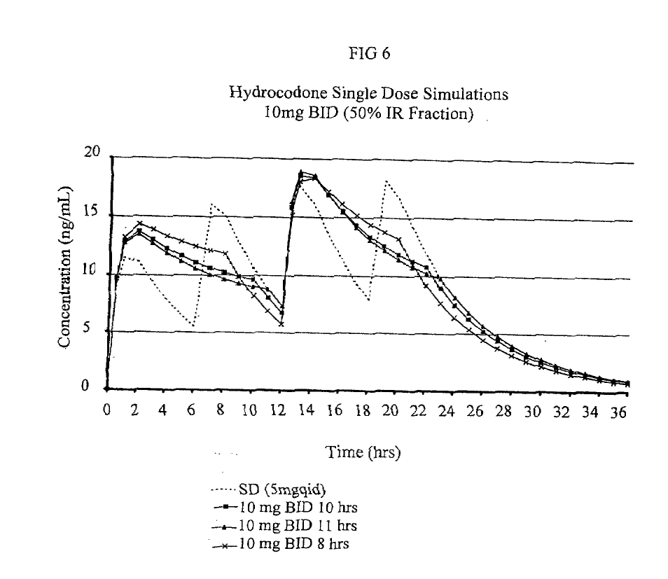

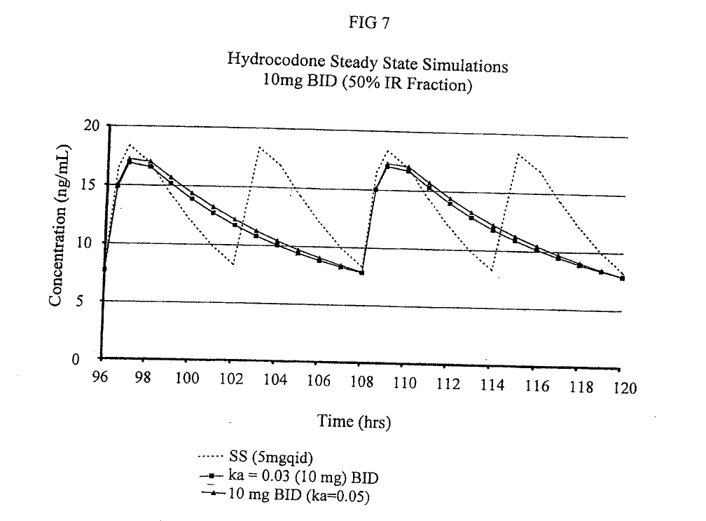

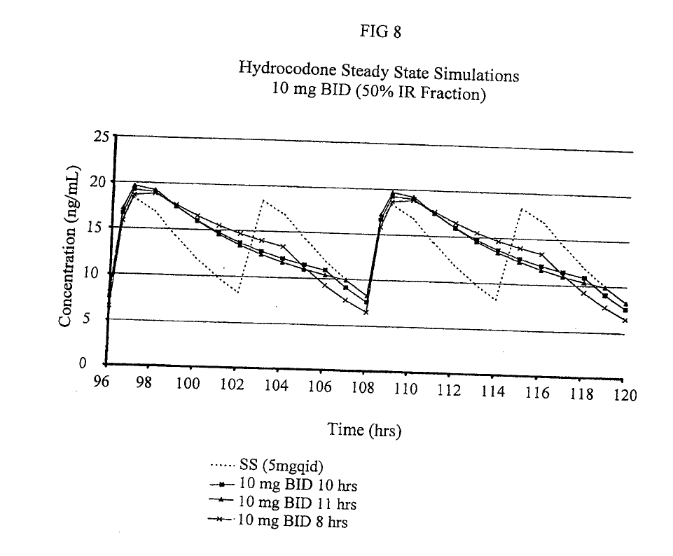

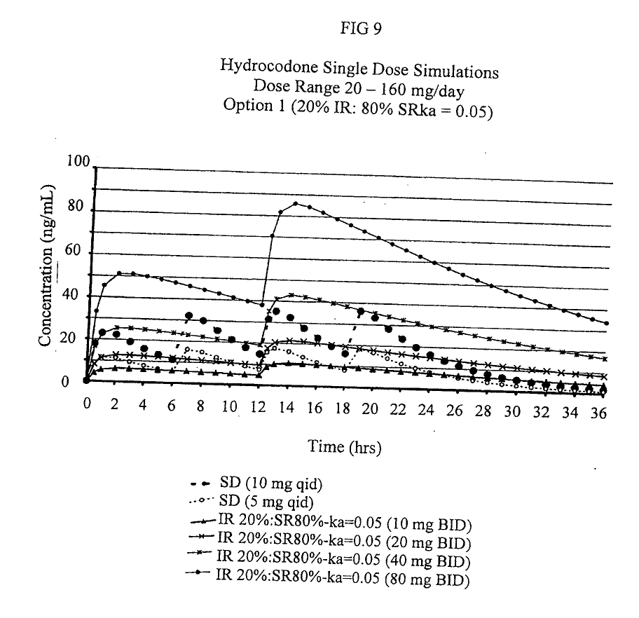

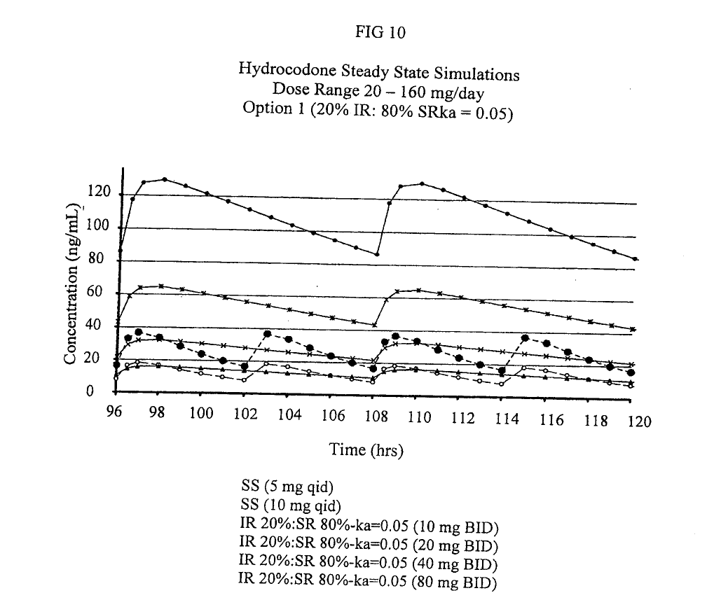

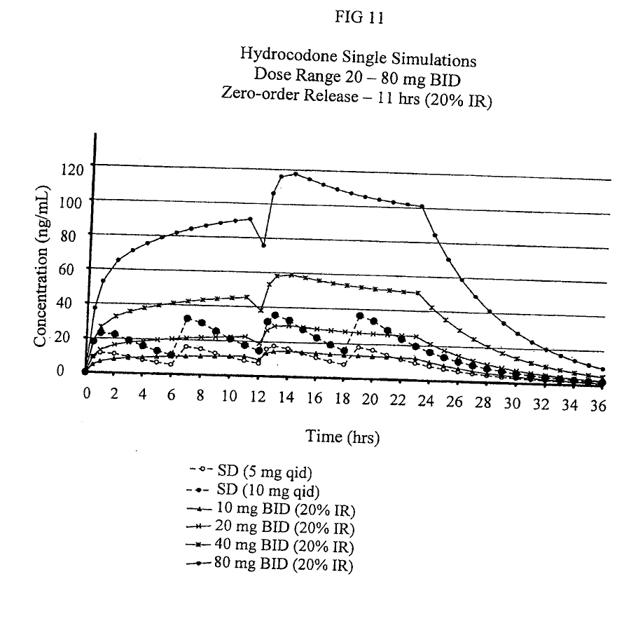

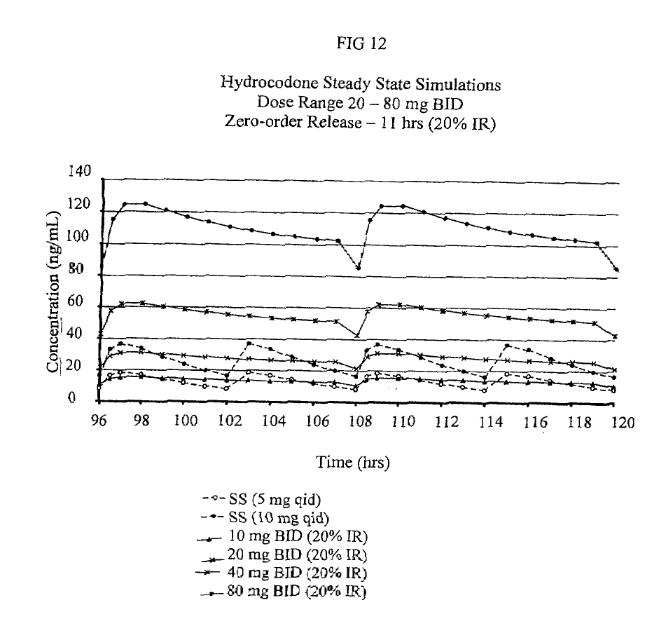

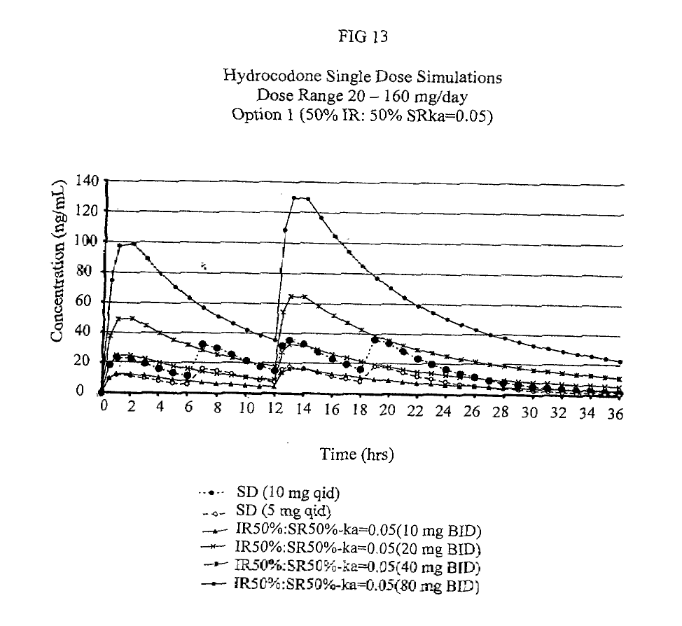

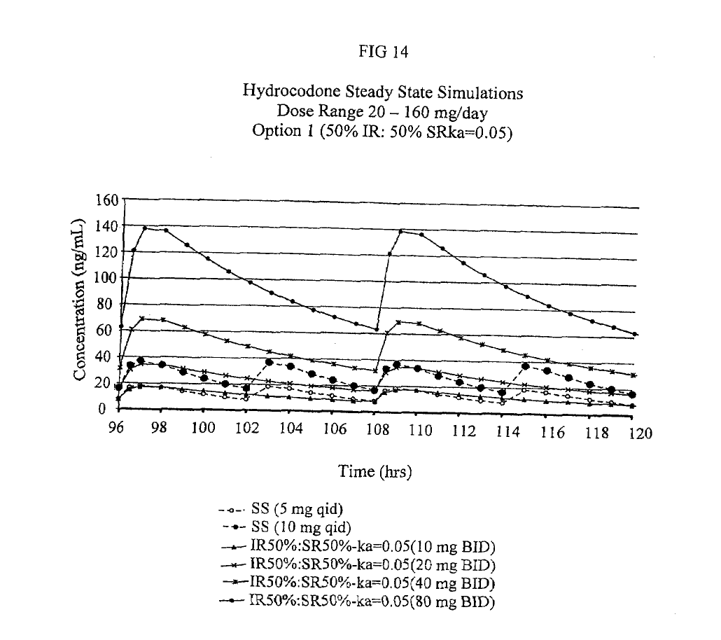

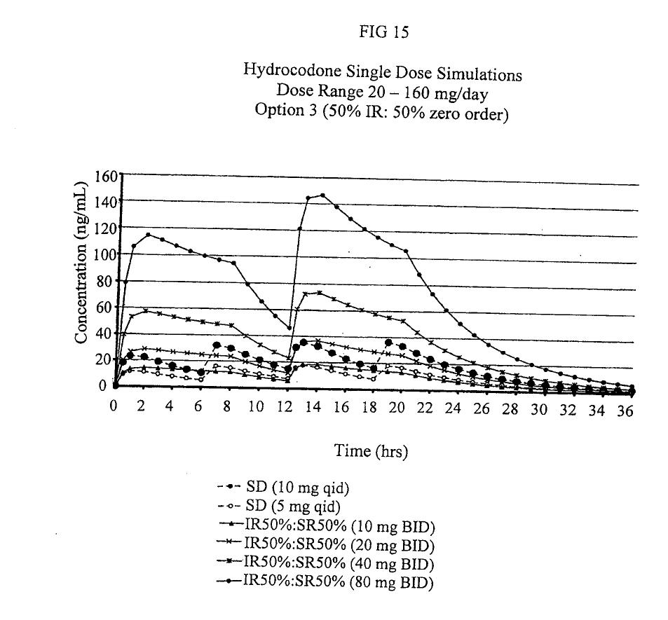

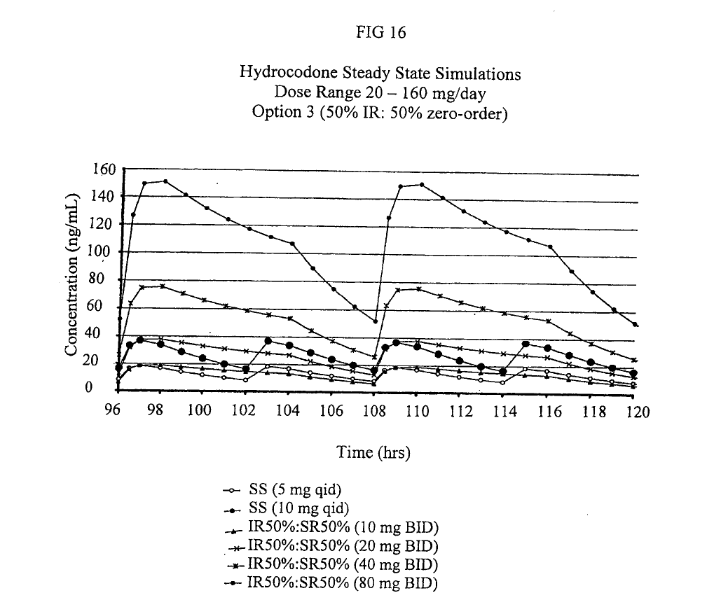

In conducting these simulations, three options were identified. Options 1 and 2 assumed a first order release and option 3 a zero-order release. Plots of the plasma concentrations of these simulations are shown in FIGS. 1 to 16.

### EXAMPLE 2

The purpose of this example is to describe preparation of a naproxen composition that can be used in the combination compositions of the invention.

To 670 g of deionized water, 30 g of hydroxypropylcellulose (Klucel Type EF; Aqualon) was dissolved using a continuous laboratory mixer. 300 g of naproxen was dispersed into the HPC solution until a homogeneous suspension was obtained. A laboratory scale media mill filled with polymeric grinding media was used in a continuous fashion until the mean particle size was approximately 200 nm as measured by laser light scattering technique, ex. Microtrak UPA.

### EXAMPLE 3

The purpose of this example is to describe preparation of a naproxen composition that can be used in the combination compositions of the invention.

To 575 g of deionized water was dissolved 25 g of polyvinylpyrrolidone (K29/32; BASF Corpl) using a continuous laboratory mixer. 400 g of naproxen was dispersed into the PVP solution until a homogenous suspension was obtained. It was processed through a laboratory scale media mill filled with polymeric grinding media in a continuous fashion until the mean particle size was approximately 200 nm as measured by laser light scattering technique, ex. MicroTrak UPA.

### EXAMPLE 4

The purpose of this example is to describe preparation of a naproxen composition that can be used in the combination compositions of the invention.

A nanoparticulate naproxen dispersion was prepared in a roller mill as follows. A 250 ml glass jar was charged with 120 ml of 1.0 mm pre-cleaned Zirconium oxide beads (Zirbeads XR, available from Zircoa Inc., having a nominal diameter of 1.0 mm), 60 g of an aqueous slurry containing 3 g naproxen (5% by weight), purchased from Sigma, St. Louis, Mo., particle size 20-30 microns, and 1.8 g (3% by weight) Pluronic F-68, purchased from BASF Fine Chemicals, Inc., as the surface stabilizer. The beads were pre-cleaned by rinsing in 1N H₂SO₄ overnight followed by several rinses with deionized water. The batch was rolled at 92 RPM for a total of 120 hours. The dispersion was stable when a portion was added to 0.1N HCl. The average particle size measured by photon correlation spectroscopy was 240-300 nm.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A composition comprising:
(a) a multiparticulate modified release composition comprising hydrocodone or a pharmaceutically acceptable salt thereof, enantiomer thereof, derivative thereof, or a mixture thereof, and comprising:
(i) a first component comprising a first population of hydrocodone comprising particles; and
(ii) at least one subsequent component, each subsequent component comprising a subsequent population of hydrocodone-comprising particles, wherein the at least one subsequent population of hydrocodone-comprising particles further comprises a modified release coating, a modified release matrix material, or a combination of a modified release coating and a modified release matrix material, such that the composition following oral delivery to a subject delivers the at least one hydrocodone, or a salt or derivative thereof, in a bimodal or multimodal manner; and
(b) a nanoparticulate naproxen composition comprising:
(i) particles of naproxen or a salt or derivative thereof having an effective average particle size of less than about 2000 nm; and
(ii) at least one surface stabilizer.

2. The composition of claim 1, wherein the multiparticulate modified release composition comprises a first component and one subsequent component.

3. The composition of claim 2, wherein the first component is an immediate release component and the subsequent component is a modified release component.

4. The composition of claim 3, wherein the modified release component comprises particles having a modified release coating.

5. The composition of claim 3, wherein the modified release component comprises a modified release matrix material.

6. The composition of claim 1, wherein for the multiparticulate modified release composition, the first population of hydrocodone-comprising particles and the at least one subsequent population of hydrocodone-comprising particles comprise the same hydrocodone.

7. The composition of claim 1, wherein for the multiparticulate modified release composition, the first population of hydrocodone-comprising particles and the at least one subsequent population of hydrocodone-comprising particles comprise different hydrocodones.

8. The composition of claim 1, wherein for the multiparticulate modified release composition, the first population of hydrocodone-comprising particles comprises two or more hydrocodones.

9. The composition of claim 1, wherein for the multiparticulate modified release composition, the at least one subsequent population of hydrocodone-comprising particles contains two or more hydrocodones.

10. The composition of claim 1, wherein for the multiparticulate modified release composition, the hydrocodone comprises substantially one optically pure enantiomer or a mixture, racemic or otherwise, of enantiomers.

11. The composition of claim 1, wherein for the multiparticulate modified release composition, at least one of the components further comprises an enhancer.

12. The composition of claim 1, wherein for the multiparticulate modified release composition, the amount of hydrocodone comprised in the first and subsequent components is the same.

13. The composition of claim 1, wherein for the multiparticulate modified release composition, the amount of hydrocodone comprised in the first component is a minor portion of the hydrocodone comprised in the composition and the amount of hydrocodone comprised in the subsequent components is a major portion of the hydrocodone comprised in the composition.

14. The composition of claim 13, wherein for the multiparticulate modified release composition, the first population of hydrocodone comprising particles comprises from about 10% to about 40% of a hydrocodone comprised in the composition and the subsequent populations of hydrocodone comprising particles comprise from about 60% to about 90% of the hydrocodone comprised in the composition

15. The composition of claim 13, wherein for the multiparticulate modified release composition, the first population of hydrocodone comprising particles comprises about 20% of the hydrocodone comprised in the composition and the subsequent populations of hydrocodone comprising particles comprise about 80% of the hydrocodone comprised in the composition.

16. The composition of claim 1, wherein for the multiparticulate modified release composition, the first and subsequent populations of hydrocodone-comprising particles have different release profiles.

17. The composition of claim 1, wherein for the multiparticulate modified release composition, the first component is an immediate release component and the at least one subsequent component is a modified release component.

18. The composition of claim 17, which:
(a) upon administration to a patient, rapidly releases the hydrocodone from the first population of hydrocodone comprising particles and releases at least about 80% of the hydrocodone from the at least one subsequent population of hydrocodone comprising particles within about 12 hours; or
(b) upon administration to a patient, rapidly releases the hydrocodone from the first population of hydrocodone comprising particles and releases at least about 80% of the hydrocodone from the at least one subsequent population of hydrocodone comprising particles within about 24 hours.

19. The composition of claim 1, wherein for the multiparticulate modified release composition, the release profile of the hydrocodone upon administration to a patient mimics the release profile of the same hydrocodone administered in the form of two or more doses of immediate release forms of the hydrocodone.

20. The composition of claim 1, wherein for the multiparticulate modified release composition, the release profile of the hydrocodone upon administration to a patient mimics the release profile of the same hydrocodone administered in the form of two or more doses of the hydrocodone in which one dose has an immediate release profile and at least one dose has a modified release profile.

21. A solid oral dosage form comprising the composition of claim 1.

22. The dosage form of claim 21, wherein the multiparticulate modified release composition comprises a blend of first and subsequent hydrocodone-comprising particles filled into hard gelatin or soft gelatin capsules.

23. The dosage form of claim 21, wherein the multiparticulate modified release composition comprises first and subsequent components which are separately and independently compressed into mini-tablets and filled into hard or soft gelatin capsules.

24. The dosage form of claim 21, wherein for the multiparticulate modified release composition, the first component is compressed into the first layer of a multilayer tablet and the at least one subsequent component is compressed into a subsequent layer of the multilayer tablet.

25. The dosage form of claim 21, wherein for the multiparticulate modified release composition, the first and subsequent components are incorporated in a rapidly dissolving dosage form.

26. The dosage form of claim 25, wherein the rapidly dissolving dosage form is a fast-melt tablet dosage form.

27. The dosage form of claim 21, wherein the hydrocodone is present in about 0.1 mg to about 1 g, or about 10 mg to about 80 mg.

28. The dosage form of claim 21 comprising:
(a) about 10 mg hydrocodone and having a mean hydrocodone Cₘₐₓ of about 8.9 ng/mL±20%;
(b) about 10 mg hydrocodone and having a hydrocodone Cₘₐₓ of about 5 to about 15 ng/mL;
(c) about 20 mg hydrocodone and having a mean hydrocodone Cₘₐₓ of about 17.9 ng/mL;
(d) about 20 mg hydrocodone and having a hydrocodone Cₘₐₓ of from about 10 to about 27 ng/mL;
(e) about 30 mg hydrocodone and having a mean hydrocodone Cₘₐₓ of about 31.7 ng/mL±20%;
(f) about 30 mg hydrocodone and having a hydrocodone Cₘₐₓ of from about 16 to about 46 ng/mL;
(g) about 40 mg hydrocodone and having a mean hydrocodone Cₘₐₓ of about 37.5 ng/mL±20%;
(h) about 40 mg hydrocodone and having a hydrocodone Cₘₐₓ of from about 28 to about 62 ng/mL;
(i) about 10 mg to about 40 mg hydrocodone and having a mean hydrocodone Tₘₐₓ of about 6 hours±20%;
(j) about 10 mg to about 40 mg hydrocodone and having a hydrocodone Tₘₐₓ of from about 4 to about 12 hours;
(k) about 10 mg hydrocodone and having a mean hydrocodone AUCₗₐₛₜ of about 109 ng*hr/mL±20%;
(l) about 10 mg hydrocodone and having a hydrocodone AUCₗₐₛₜ of from about 73 to about 179 ng*hr/mL;
(m) about 20 mg hydrocodone and having a mean hydrocodone AUCₗₐₛₜ of about 212.9 ng*hr/mL±20%;
(n) about 20 mg hydrocodone and having a hydrocodone AUCₗₐₛₜ of from about 130 to about 377 ng*hr/mL;
(o) about 30 mg hydrocodone and having a mean hydrocodone AUCₗₐₛₜ of about 392.5 ng*hr/mL±20%;
(p) about 30 mg hydrocodone and having a hydrocodone AUCₗₐₛₜ of from about 177 to about 671 ng*hr/mL;
(q) about 40 mg hydrocodone and having a mean AUCₗₐₛₜ of about 464.6 ng*hr/mL, +20%;
(r) about 40 mg hydrocodone and having a hydrocodone AUCₗₐₛₜ of from about 321 to about 712 ng*hr/mL.

29. The composition of claim 1, wherein the nanoparticulate naproxen particle is selected from the group consisting of a crystalline phase, an amorphous phase, a semi-crystalline phase, a semi amorphous phase, and mixtures thereof.

30. The composition of claim 1, wherein the effective average particle size of the naproxen particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

31. The composition of claim 1, wherein the composition is formulated:
(a) for administration selected from the group consisting of parental injection, oral administration in solid, liquid, or aerosol form, vaginal, nasal, rectal, otically, ocular, local, buccal, intracistemal, intraperitoneal, and topical administration;
(b) into a dosage form selected from the group consisting of liquid dispersions, gels, sachets, solutions, aerosols, ointments, tablets, capsules, creams, and mixtures thereof;
(c) into a dosage form selected from the group consisting of controlled release formulations, fast melt formulations, lyophilized formulations, delayed release formulations, extended release formulations, pulsatile release formulations, and mixed immediate release and controlled release formulations; or
(d) any combination thereof.

32. The composition of claim 1, wherein:
(a) naproxen is present in an amount consisting of from about 99.5% to about 0.001%, from about 95% to about 0.1%, or from about 90% to about 0.5%, by weight, based on the total combined dry weight of naproxen and at least one surface stabilizer, not including other excipients;
(b) the at least one surface stabilizer is present in an amount of from about 0.5% to about 99.999% by weight, from about 5.0% to about 99.9% by weight, or from about 10% to about 99.5% by weight, based on the total combined dry weight of naproxen and at least one surface stabilizer, not including other excipients; or
(c) a combination thereof.

33. The composition of claim 1, wherein the surface stabilizer is selected from the group consisting of a nonionic surface stabilizer, an anionic surface stabilizer, a cationic surface stabilizer, a zwitterionic surface stabilizer, and an ionic surface stabilizer.

34. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₇ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

35. The composition of claim 1, wherein the composition does not produce significantly different naproxen absorption levels when administered under fed as compared to fasting conditions.

36. The composition of claim 1, wherein administration of the composition to a subject in a fasted state is bioequivalent to administration of said composition to a subject in a fed state.

37. The composition of claim 36, wherein “bioequivalency” is established by:
(a) a 90% Confidence Interval of between 0.80 and 1.25 for both Cₘₐₓ and AUC; or
(b) a 90% Confidence Interval of between 0.80 and 1.25 for AUC and a 90% Confidence Interval of between 0.70 to 1.43 for Cₘₐₓ.

38. The composition of claim 1, wherein:
(a) the Tₘₐₓ of naproxen or a salt or derivative thereof, when assayed in the plasma of a mammalian subject following administration, is less than the Tₘₐₓ for a non-nanoparticulate composition of the same naproxen, administered at the same dosage;
(b) the Cₘₐₓ of naproxen or a salt or derivative thereof, when assayed in the plasma of a mammalian subject following administration, is greater than the Cₘₐₓ for a non-nanoparticulate composition of the same naproxen, administered at the same dosage;
(c) the AUC of naproxen or a salt or derivative thereof, when assayed in the plasma of a mammalian subject following administration, is greater than the AUC for a non-nanoparticulate composition of the same naproxen, administered at the same dosage; or
(d) any combination thereof.

39. A method for the treatment of pain comprising administering a therapeutically effective amount of a composition according to claim 1.
